(19) United States
(12) Patent Application Publication
(10) Pub. No.: US 2019/0100518 A1
(43) Pub. Date: Apr. 4, 2019

(54) INDOLE STRUCTURE-SELECTIVE CROSSLINKING AGENT AND COMPOSITE IN WHICH SAME IS USED

(71) Applicant: The University of Tokyo, Bunkyo-ku (JP)

(72) Inventors: Motomu KANAI, Bunkyo-ku (JP); Kounosuke OISAKI, Bunkyo-ku (JP); Yohei SEKI, Bunkyo-ku (JP); Daisuke SASAKI, Bunkyo-ku (JP); Takashi ISHIYAMA, Bunkyo-ku (JP)

(73) Assignee: The University of Tokyo, Bunkyo-ku (JP)

(21) Appl. No.: 16/083,332

(22) PCT Filed: Mar. 8, 2017
(86) PCT No.: PCT/JP2017/009315

§ 371 Date: Sep. 7, 2018

Related U.S. Application Data
(60) Provisional application No. 62/305,759, filed on Mar. 9, 2016.

(51) Int. Cl. C07D 451/14; C08L 89/00

(52) U.S. Cl. CPC C07D 451/14; A61K 47/65; C08L 2312/00; C08L 89/00

(57) ABSTRACT

The present invention relates to a cross-linking agent for cross-linking an indole-structure-containing molecule to a desired molecule, the cross-linking agent containing, as an effective component, a radical compound having an N-oxy radical group and a group capable of bonding to the desired molecule.

### CROSS-REFERENCE TO RELATED APPLICATIONS

The present patent application claims the priority based on U.S. provisional application No. 62/305,759 (filing date: Mar. 9, 2016), which is a provisional patent application previously applied in the United States. The entire content disclosed in this previous provisional patent application is incorporated herein by citation.

### TECHNICAL FIELD

The present invention relates to an indole structure-selective cross-linking agent and a conjugate using it.

### BACKGROUND ART

By modification of a biologically active substance, the biologically active substance can have a new function that has not been originally retained in the substance. For example, protein modification is expected to be a tool that may enable development of various application fields such as biological control, analysis of life phenomena, creation of biocompatible materials, and novel therapeutic methods.

In modification reaction of a protein, from the viewpoint of giving a new action function while maintaining the action function of the protein itself, it is preferred to carry out the modification site-selectively such that the original structure and function of the protein can be maintained. However, protein modification reaction has a problem that, since lysine residues are generally present in a large amount on the protein surface, the number and the positions of the lysines to be modified cannot be easily controlled. Furthermore, since cysteine residues are often forming a disulfide bond (—S—S—) that contributes to the higher-order structure of the protein, when cysteine residues are exposed by reduction of the disulfide bond, the higher-order structure of the protein is affected by this process, which is problematic (Non-patent Document 1).

On the other hand, tryptophan residues are present only in a small amount on the protein surface. It is therefore thought that use of the amino acid residue having an electron-rich aromatic ring side chain as a target of modification reaction may contribute to solutions of the problems in the conventional techniques described above.

However, since tryptophan residues are poorly reactive, reactions targeting tryptophan residues are limited, and conventional methods therefor require use of a harmful transition metal catalyst or non-biocompatible strict conditions. Thus, there is a problem that use of the reaction product is limited (Non-patent Document 2).

Use of keto-azabicyclo[3.3.1]nonane N-oxyl (keto-ABNO) as an alcohol oxidation catalyst has been reported (Non-patent Document 3 and Patent Document 1). However, use of keto-ABNO in a modification reaction targeting the indole side chain of a tryptophan residue in a protein has not been reported at all.

### PRIOR ART DOCUMENTS

### Non-Patent Documents

Non-patent Document 1: Hogg, P. J. Trends Biochem. Sci. 2003, 28, 210-214
Non-patent Document 2: Antos, J.; Francis, M. B. J. Am. Chem. Soc. 2004, 126, 10256-10257
Non-patent Document 3: Sonobe et al., Chem. Sci., 2012, 3, 3249-3255

### Patent Document

Patent Document 1: JP 48030745 B

### SUMMARY OF THE INVENTION

As a result of intensive study, the present inventors discovered that use of keto-ABNO as a cross-linking agent allows simple and selective modification targeting the indole side chain of a tryptophan residue in a protein without application of a transition metal catalyst or non-biocompatible conditions. Further, the present inventors discovered that keto-ABNO and derivatives thereof dissolve in water and can be preferably used also for modification reaction in an aqueous solvent. The present invention is based on such discoveries.

Accordingly, an object of the present invention is to provide a cross-linking agent that allows site-selective modification reaction of a molecule having an indole structure without requiring a transition metal catalyst or non-biocompatible conditions, and a conjugate using it.

By the present invention, the following inventions are provided.

(1) A cross-linking agent for cross-linking an indole-structure-containing molecule to a desired molecule, the cross-linking agent comprising a compound having an N-oxy radical group and a group capable of bonding to the desired molecule.
(2) The cross-linking agent according to (1), wherein the N-oxy radical group is a dialkylaminooxy radical group.
(3) The cross-linking agent according to (1) or (2), comprising as an effective component an ABNO derivative represented by Formula (I):

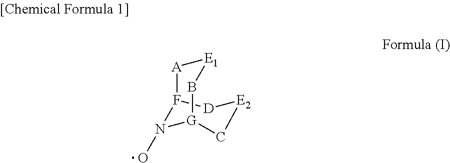

(wherein in the formula,

at least one of the groups A, B, C, D, E₁, and E₂ is a group capable of bonding to the desired molecule;

A, B, C, D, E₁, and E₂ each independently represent CR1R2; C═CR3R4; C═O; C═S; C═NR5; NR5; SIR6R7; an oxygen atom; or a heteroatom other than a nitrogen atom, silicon atom, or oxygen atom;

F and G each represent CR8 or a nitrogen atom;

R1, R2, R3, R4, R5, R6, R7, and R8 each independently represent a hydrogen atom; halogen atom; heteroatom group; C₁-C₃₀ alkyl group which is optionally substituted, C₂-C₃₀ alkenyl group which is optionally substituted, C₂-C₃₀ alkynyl group which is optionally substituted, C₆-C₃ aryl group which is optionally substituted, C₄-C₃₀ heteroaryl group which is optionally substituted, C₇-C₃₀ aralkyl group which is optionally substituted, or C₃-C₃₀ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₇-C₃₀ aralkyloxy group which Is optionally substituted, or C₃-C₃₀ cycloalkyloxy group which is optionally substituted; or polyalkyleneoxy group which is optionally substituted;

with the proviso that R5 is not a halogen atom;

R1, R2, R3, R4, R5, R6, R7, and R8 are each optionally a reactive functional group; and

E₁ and E₂ optionally together form a —CH(CH))ₘCH— group which is optionally substituted, wherein m represents an integer of 0 to 12).

(4) The cross-linking agent according to (3), wherein

A, B, C, and D each represent CH₂; and

one of E₁ and E₂ represents CH₂, an oxygen atom, or a sulfur atom, and the other represents CR1R2, C═CR3R4, C═O, C═S, C═NR5, NR5, or SiR6R7; or

E₁ and E₂ together form a —CH(CH₂)ₘCH— group which is optionally substituted.

(5) The cross-linking agent according to (3) or (4), wherein the reactive functional group is a functional group having, as a group or a part of a group, an alcohol group, epoxy group, acetal group, orthoester group, ester group, carbonyl group, carboxyl group, anhydrous carboxylic acid group, amide group, imidate group, amino group, imino group, aziridine group, diazo group, azide group, amidyl group, guanidyl group, hydrazyl group, hydrazone group, alkoxyamino group, oxime group, carbonate group, carbamate group, sulfhydryl group, ether group, imide group, thioester group, thioamide group, isothiocyano group, thioether group, disulfide group, halogen group, isocyano group, isocyanate group, oxazirine group, diaziridine group, sulfonyl group, sulfone group, sulfoxide group, sulfonimide group, seleno group, silyl group, boryl group, stannyl group, phosphine group, phosphine oxide group, phosphate group, phosphoric acid ester group, phosphoric acid amide group, methylene group, alkenyl group, or alkynyl group, or a combination thereof.
(6) The cross-linking agent according to any one of (3) to (5), wherein

A, B, C, and D each represent CH₂; and

one of E₁ and E₂ represents CH₂ or an oxygen atom, and the other represents C═O, CHNH₂, CH(CO)NH₂, or C═NOH.

(7) The cross-linking agent according to any one of (3) to (6), wherein one of E₁ and E₂ represents CH₂, and the other represents C═O, CHNH₂, CH(CO)NH₂, or C═NOH.
(8) The cross-linking agent according to any one of (3) to (7), for bonding the desired molecule to the indole structure in the indole-structure-containing molecule.
(9) The cross-linking agent according to (8), wherein the indole structure is represented by Formula (II):

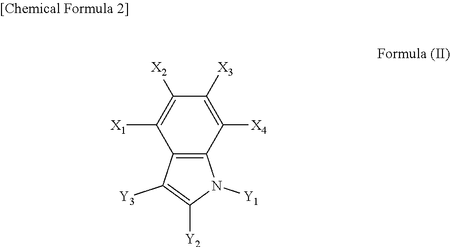

(wherein in the formula,

X₁, X₂, X₃, X₄, Y₂, and Y₃ each independently represent a hydrogen atom; or a functional group capable of substituting a hydrogen atom on an indole ring;

the functional group capable of substituting a hydrogen atom on an indole ring is optionally a halogen atom; heteroatom group; C₁-C₃₀ alkyl group which is optionally substituted, C₂-C₃₀ alkenyl group which is optionally substituted, C₂-C₃₀ alkynyl group which is optionally substituted, C₆-C₃₀ aryl group which Is optionally substituted, C₄-C₃₀ heteroaryl group which is optionally substituted, C₇-C₃₀ aralkyl group which is optionally substituted, or C₃-C₃₀ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which Is optionally substituted, C₄-C₃₀ heteroaryloxy group which is optionally substituted, C₇-C₃₀ aralkyloxy group which is optionally substituted, or C₃-C₃₀ cycloalkyloxy group which Is optionally substituted; or polyalkyleneoxy group which is optionally substituted;

at least two of the groups X₁, X₂, X₃, X₄, Y₂, and Y₃ optionally together form a 4- to 10-membered ring; and

Y₁ represents a hydrogen atom, or a functional group capable of substituting hydrogen on nitrogen on an indole ring).

(10) The cross-linking agent according to (9), wherein

X₁, X₂, X₃, X₄, Y₁, and Y₂ each represent a hydrogen atom;

Y₃ represents —(CH₂)_{□}CGF₁F₂;

O represents an integer of 0 to 10;

G represents hydrogen, alkyl group, alkyloxy group, or aryl group;

F₁ and F₂ each independently represent —NR9COZ₁ or —CONR10Z₂;

R9 and R10 each independently represent a hydrogen atom; or a functional group capable of substituting a hydrogen atom on an amide group;

the functional group capable of substituting a hydrogen atom on an amide group is optionally a C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, or C₃-C₃ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₇-C₃₀ aralkyloxy group which Is optionally substituted, or C₃-C₃₀ cycloalkyloxy group which is optionally substituted; or polyalkyleneoxy group which is optionally substituted; and

Z₁ and Z₂ are each a natural product, a synthetic product, or a linked body thereof.

(11) The cross-linking agent according to any one of (1) to (10), wherein the Indole-structure-containing molecule is a peptide containing tryptophan.
(12) The cross-linking agent according to any one of (1) to (11), wherein the desired molecule is a drug, toxin, labeling substance, fiber, peptide, protein, nucleic acid, cell, organic electronic material, or polymer material.
(13) A conjugate cross-linked through the compound recited in (1) or (2) or the ABNO derivative recited in any one of (3) to (12).
(14) The conjugate according to (13), which is a conjugate formed by bonding of a desired molecule to an indole structure in an indole-structure-containing molecule, wherein the indole structure is cross-linked to the desired molecule through the ABNO derivative.
(15) The conjugate according to (13) or (14), comprising the structure of Formula (III):

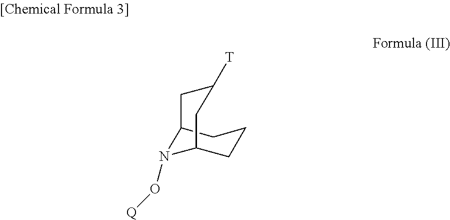

[wherein in the formula,

T is the desired molecule linked to the condensed bicyclic structure in Formula (III);

Q is a group represented by either Formula (IV) or Formula (V):

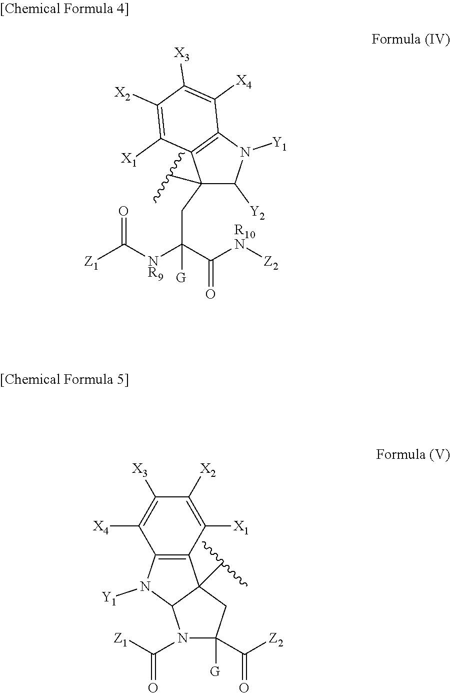

(wherein in the formulae,

X₁, X₂, X₃, X₄, and Y₂ are each a hydrogen atom; or a functional group capable of substituting a hydrogen atom on an indole ring;

Y₁ represents a functional group capable of substituting a hydrogen atom on a nitrogen atom;

R10 and R11 are each a hydrogen atom; or a capable group capable of substituting a hydrogen atom on an amide group;

at least two of the groups X₁, X₂, X₃, and X₄ optionally together form a 4- to 10-membered ring;

G represents hydrogen, alkyl group, alkyloxy group, or aryl group; and

Z₁ and Z₂ are each a natural product, a synthetic product, or a linked body thereof)].

(16) The conjugate according to (15), wherein X₁, X₂, X₃, X₄, and G each represent a hydrogen atom.
(17) A method for producing a conjugate in which a desired molecule is bound to an indole structure in an indole-structure-containing molecule, the method comprising the step of cross-linking the indole-structure-containing molecule to the desired molecule through a compound having an N-oxy radical group and a group capable of bonding to the desired molecule.
(18) The production method according to (17), comprising the step of allowing the desired molecule to act on the compound, and then allowing the compound, to which the desired molecule is bound, to act on the indole structure in the indole-structure-containing molecule.
(19) The production method according to (17), comprising the step of allowing the compound to act on the indole structure in the indole-structure-containing molecule, and then allowing the desired molecule to act on the compound bound to the indole-structure-containing molecule.
(20) The method according to any one of (17) to (19), wherein the cross-linking reaction is carried out in an aqueous solvent.

According to the present invention, by using an ABNO derivative as a cross-linking agent, a cross-linking reaction selective to a molecule having an indole structure can be simply carried out at room temperature in underwater conditions without using a transition metal catalyst or performing a pretreatment such as oxidation or reduction. Since the cross-linking agent of the present Invention is capable of selectively reacting with an indole structure in a protein under mild conditions such as in an aqueous solvent, the agent is especially advantageous when a cross-linked conjugate of a protein is to be stably formed while the spatial structure and the function of the protein are maintained.

### BRIEF DESCRIPTION OF THE DRAWINGS

FIG. 1 shows a diagram illustrating the crystal structure of a conjugate between keto-ABNO and lysozyme according to the result of X-ray crystallography.

FIG. 2 shows a photograph showing the results of SDS-PAGE for a test group (keto-ABNO-lysozyme conjugate) and a control group.

FIG. 3 shows photographs showing the result of a dot blot assay of a conjugate between keto-ABNO-fluorescein methyl ester (FL) and an anti-amyloid B antibody 6E10.

FIG. 4 shows charts showing the results of HPLC of each ABNO and O-acyl-isoAβ₁₋₄₂-Trp.

FIG. 5 shows a chart showing the results on the circular dichroism (CD) spectra for a test group (keto-ABNO-lysozyme conjugate) and a control group (lysozyme alone).

FIG. 6A and FIG. 6B show charts showing the results of LC analysis of a test group (keto-ABNO-lysozyme conjugate) and a control group (phenylmalelmide-lysozyme conjugate prepared by a cysteine modification method), respectively.

FIG. 7 shows a chart showing the results on the circular dichroism (CD) spectra for a test group (keto-ABNO-lysozyme conjugate) and control groups A to C.

FIG. 8 shows a graph showing the aggregation-suppressing effect of β2-microglobulin in each of a test group (keto-ABNO-β2-microglobulin conjugate) and control groups A to C.

### SPECIFIC DESCRIPTION OF THE INVENTION

### Definitions

Unless otherwise specified, the terms “alkyl”, “alkenyl”, “alkynyl”, and “aralkyl” in the present description mean alkyl, alkenyl, and alkynyl, respectively, having a linear, branched, or cyclic shape, or having a combined shape thereof. In cases of a cyclic alkyl, its carbon number is at least three.

In the present description, the term “halogen atom” is fluorine, chlorine, bromine, or iodine, preferably fluorine, chlorine, or bromine.

In the present description, the term “heteroatom” means an atom having a valence of not less than two other than carbon or hydrogen. “Heteroatom” is preferably an oxygen atom, nitrogen atom, or sulfur atom.

In the present description, the term “heteroatom group” means a substituent having a heteroatom as a part of the group. The “heteroatom group” Is preferably a hydroxyl group, amino group, or thiol group, more preferably a hydroxyl group or amino group, still more preferably a hydroxyl group.

In the present description, the term “optionally substituted” as used for an alkyl group means that one or more hydrogen atoms on the alkyl group may be substituted by one or more substituents (which may be the same or different). It would be evident to those skilled in the art that the maximum number of substituents can be determined depending on the number of hydrogen atoms that can be substituted on the alkyl group. These also apply to functional groups other than alkyl groups.

### Cross-Linking Agent

The cross-linking agent of the present Invention Is characterized in that it contains as an effective component a compound having an N-oxy radical group and a group capable of bonding to a desired molecule. It is surprising that a radical oxygen at an end of such an ABNO derivative can selectively bond to an indole structure.

In the radical compound of the present invention, the N-oxy radical group Is preferably a dialkylaminooxy radical group.

In a more preferred mode, the radical compound of the present invention is an ABNO derivative represented by Formula (I).

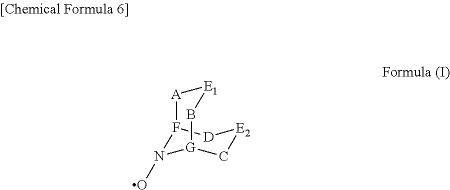

(wherein in the formula,

at least one of the groups A, B, C, D, E₁, and E₂ is a group capable of bonding to a desired molecule;

A, B, C, D, E₁, and E₂ each independently represent CR1R2; C═CR3R4; C═O; C═S; C═NR5; NR5; SiR6R7; an oxygen atom; or a heteroatom other than a nitrogen atom, silicon atom, or oxygen atom;

F and G each represent CR8 or a nitrogen atom;

R1, R2, R3, R4, R5, R6, R7, and R8 each independently represent a hydrogen atom; halogen atom; heteroatom; C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, or C₃-C₃₀ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₇-C₃₀ aralkyloxy group which is optionally substituted, or C₃-C₃₀ cycloalkyloxy group which Is optionally substituted; or polyalkyleneoxy group which is optionally substituted;

with the proviso that R5 is not a halogen atom;

R1, R2, R3, R4, R5, R6, R7, and R8 are each optionally a reactive functional group; and

E₁ and E₂ optionally together form a —CH(CH)ₘCH— group which Is optionally substituted, wherein m represents an integer of 0 to 12).

The carbon number of the alkyl group in each of R1, R2, R3, R4, R6, R7, and R8 is preferably 1 to 20. Specific examples of the C₁-C₃ alkyl group include methyl, ethyl, n-propyl, iso-propyl, n-butyl, iso-butyl, sec-butyl, tert-butyl, n-pentyl, n-hexyl, n-octyl, n-decyl, n-dodecyl, n-octadecyl, and n-icosyl.

The carbon number of the alkenyl group in each of R1, R2, R3, R4, R6, R7, and R8 is preferably 2 to 15. Preferred examples of the C₂-C₃₀ alkenyl group include vinyl, allyl, 3-butenyl, 4-pentenyl, 5-hexenyl, 6-heptenyl, and 7-octenyl.

The carbon number of the alkynyl group in each of R1, R2, R3, R4, R6, R7, and R8 is preferably 2 to 15. Preferred examples of the C₂-C₃n alkynyl group include ethynyl, 2-propynyl, 3-butynyl, 4-pentynyl, 5-hexynyl, 6-heptynyl, and 7-octynyl.

The carbon number of the aralkyl group in each of R1, R2, R3, R4, R6, R7, and R8 is preferably 7 to 15. Preferred examples of the C₇-C₃₀ aralkyl group include benzyl and 1-phenetyl.

The carbon number of the aryl group in each of R1, R2, R3, R4, R6, R7, and R8 is preferably 6 to 15. Preferred examples of the C₆-C₃₀ aryl group include phenyl, 1-naphthyl, and 2-naphthyl.

The carbon number of the heteroaryl group in each of R1, R2, R3, R4, R6, R7, and R8 is preferably 6 to 15. Preferred examples of the C₄-C₃₀ heteroaryl group include 2-pyridyl, 3-pyridyl, 4-pyridyl, 2-thiophenyl, and 2-furyl.

The carbon number of the cycloalkyl group in each of R1, R2, R3, R4, R6, R7, and R8 is preferably 3 to 15. Preferred examples of the C₃-C₃₀ cycloalkyl group include cyclopropyl, cyclobutyl, cyclopentyl, and cyclohexyl.

The alkyl group, alkenyl group, alkynyl group, aryl group, heteroaryl group, aralkyl group, or cycloalkyl group constituting part of the alkyloxy group, alkenyloxy group, alkynyloxy group, aryloxy group, heteroaryloxy group, aralkyloxy group, or cycloalkyloxy group, respectively, in each of R1, R2, R3, R4, R5, R6, R7, and R8 may be selected from the same groups as those for the alkyl group, alkenyl group, alkynyl group, aryl group, heteroaryl group, aralkyl group, or cycloalkyl group, respectively, in each of R1, R2, R3, R4, R5, R6, R7, and RB.

The polyalkyleneoxy group in each of R1, R2, R3, R4, R5, R6, R7, and R8 is preferably a polyethyleneoxy group, more preferably a —(CH₂CH₂O)a- group (wherein a is an Integer of 1 to 10), more preferably a —(CH₂CH₂O)a- group (wherein a is 4).

Examples of the substituent in each of R1, R2, R3, R4, R5, R6, R7, and R8 include a hydrogen atom; halogen atom; heteroatom group (preferably a hydroxyl group or the like); C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, and C₃-C₃₀ cycloalkyl group which is optionally substituted; C₁-C₃₀ alkyloxy group which Is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₁-C₃₀ aralkyloxy group which is optionally substituted, and C₃-C₃₀ cycloalkyloxy group which is optionally substituted; polyalkyleneoxy group which is optionally substituted; and reactive functional group (with the proviso that R5 is not a halogen atom). Preferred examples of the substituent include the reactive functional groups described later and halogen atoms. The number of the substituent(s) and the position(s) of substitution are not limited.

The reactive functional group as a group or a part (substituent) of the group in each of R1, R2, R3, R4, R5, R6, R7, and R8 may be appropriately selected by those skilled in the art described in, for example, Greg T. Hermanson “Bloconjugate Techniques, third edition”, taking into account giving of a desired reactivity to the compound of Formula (I) and formation of a cross-link with the desired molecule. The reactive functional group is preferably a functional group capable of forming a cross-link with the desired molecule. Preferred examples of the reactive functional group include functional groups having, as a group or a part of a group, an alcohol group, epoxy group, acetal group, orthoester group, ester group, carbonyl group, carboxyl group, anhydrous carboxylic acid group, amide group, imidate group, amino group, imino group, aziridine group, diazo group, azide group, amidyl group, guanidyl group, hydrazyl group, hydrazone group, alkoxyamino group, oxime group, carbonate group, carbamate group, sulfhydryl group, ether group, imide group, thioester group, thioamide group, isothiocyano group, thioether group, disulfide group, halogen group, isocyano group, isocyanate group, oxazirine group, diaziridine group, sulfonyl group, sulfone group, sulfoxide group, sulfonimide group, seleno group, silyl group, boryl group, stannyl group, phosphine group, phosphine oxide group, phosphate group, phosphoric acid ester group, phosphoric acid amide group, methylene group, alkenyl group, or alkynyl group. More preferred examples of the reactive functional group include functional groups having, as a group or a part of a group, an alcohol group, epoxy group, ester group, carbonyl-containing group, carboxyl group, anhydrous carboxylic acid group, amide group, amino group, azide group, hydrazone group, oxime group, carbonate group, carbamate group, sulfhydryl group, maleimide group, thioester group, thioamide group, isothiocyano group, thioether group, isocyano group, isocyanate group, oxazirine group, azide group, methanesulfonyl group, p-toluenesulfonyl group, methylene group, alkenyl group, or alkynyl group, or a combination thereof. Preferred examples of the reactive functional group having a methylene group, alkenyl group, or alkynyl group may include those having, as a group or a part of a group, a C₁-C₃₀ alkyl group, C₂-C₃₀ alkenyl group, C₂-C₃₀ alkynyl group, C₆-C₃₀ aryl group, C₄-C₃₀ heteroaryl group, C₇-C₃₀ aralkyl group, C₃-C₃₀ cycloalkyl group, C₁-C₃₀ alkyloxy group, C₂-C₃₀ alkenyloxy group, C₂-C₃₀ alkynyloxy group, C₆-C₃₀ aryloxy group, C₄-C₃₀ heteroaryloxy group, C₇-C₃₀ aralkyloxy group, C₃-C₃₀ cycloalkyloxy group, or the like. The present invention also Includes such modes.

In Formula (I), the combination of the substituent(s) and/or functional group(s) (including a reactive functional group(s)) In R1, R2, R3, R4, R5, R6, R7, and R8 is selected such that at least one of the groups A, B, C, D, E₁, and E₂ is a group capable of bonding to the desired molecule. Thus, at least one of the substituents in R1, R2, R3, R4, R5, R6, R7, and RB is preferably a functional group capable of cross-linking with the desired molecule. The type of such a functional group is appropriately selected by those skilled in the art depending on reactivity with the desired molecule, and/or the like.

In one preferred mode, each of R1, R2, R3, R5, R4, R6, R7, and R8 is preferably a C₁-C₃₀ alkyl group which is optionally substituted, or a C₂-C₃ alkenyl group which is optionally substituted, more preferably an n-butyl group, n-pentyl group, n-hexyl group, allyl group, or 3-butenyl group.

In another preferred mode, at least one of R1, R2, R3, R5, R4, R6, R7, and R8 is preferably a polyalkyleneoxy group. More preferably, R5 is a polyalkyleneoxy group. The polyalkyleneoxy group is preferably substituted. Preferred examples of the substituent Include allyl, vinyl, phenyl, naphthyl, pyridyl, and triazole.

In a preferred mode, A, B, C, and D in the ABNO derivative in the present invention are each independently CR1R2, preferably CH₂ or CHR1. In the CHR1 represented by each of A, B, C, and D, R1 is a reactive functional group, more preferably a functional group having, as a group or a part of a group, an alcohol group, epoxy group, acetal group, orthoester group, ester group, carbonyl group, carboxyl group, anhydrous carboxylic acid group, amide group, imidate group, amino group, imino group, aziridine group, diazo group, azide group, amidyl group, guanidyl group, hydrazyl group, hydrazone group, alkoxyamino group, oxime group, carbonate group, carbamate group, sulfhydryl group, ether group, imide group, thioester group, thioamide group, isothiocyano group, thioether group, disulfide group, halogen group, isocyano group, isocyanate group, oxazirine group, diaziridine group, sulfonyl group, sulfone group, sulfoxide group, sulfonimide group, seleno group, silyl group, boryl group, stannyl group, phosphine group, phosphine oxide group, phosphate group, phosphoric acid ester group, phosphoric acid amide group, methylene group, alkenyl group, or alkynyl group, still more preferably a functional group having, as a group or a part of a group, an alcohol group, epoxy group, ester group, carbonyl-containing group, carboxyl group, anhydrous carboxylic acid group, amide group, amino group, azide group, hydrazone group, oxime group, carbonate group, carbamate group, sulfhydryl group, maleimide group, thioester group, thioamide group, isothiocyano group, thioether group, isocyano group, isocyanate group, oxazirine group, azide group, methanesulfonyl group, p-toluenesulfonyl group, methylene group, alkenyl group, or alkynyl group, or a combination thereof.

In another preferred mode, E₁ and E₂ in the ABNO derivative in the present invention are each independently CR1R2, C═CR3R4, C═O, C═S, C═NR5, NR5, SIR6R7, or a heteroatom other than a nitrogen atom, preferably C═O, C═NR5, CHR1, CH₂, or an oxygen atom. Here, in the C═NR5 and the CHR1 represented by each of E₁ and E₂, R1 and R5 are each preferably a reactive functional group, more preferably a functional group having, as a group or a part of a group, an alcohol group, epoxy group, acetal group, orthoester group, ester group, carbonyl group, carboxyl group, anhydrous carboxylic acid group, amide group, imidate group, amino group, imino group, aziridine group, diazo group, azide group, amidyl group, guanidyl group, hydrazyl group, hydrazone group, alkoxyamino group, oxime group, carbonate group, carbamate group, sulfhydryl group, ether group, imide group, thioester group, thioamide group, isothiocyano group, thioether group, disulfide group, halogen group, isocyano group, isocyanate group, oxazirine group, diaziridine group, sulfonyl group, sulfone group, sulfoxide group, sulfonimide group, seleno group, silyl group, boryl group, stannyl group, phosphine group, phosphine oxide group, phosphate group, phosphoric acid ester group, phosphoric acid amide group, methylene group, alkenyl group, or alkynyl group, still more preferably a functional group having, as a group or a part of a group, an alcohol group, epoxy group, ester group, carbonyl-containing group, carboxyl group, anhydrous carboxylic acid group, amide group, amino group, azide group, hydrazone group, oxime group, carbonate group, carbamate group, sulfhydryl group, maleimide group, thioester group, thioamide group, isothiocyano group, thioether group, isocyano group, isocyanate group, oxazirine group, azide group, methanesulfonyl group, p-toluenesulfonyl group, methylene group, alkenyl group, or alkynyl group, or a combination thereof.

Alternatively, E₁ and E₂ may together form a —CH(CH₂)ₘCH— group. Here, m Is preferably an integer of 0 to 12, more preferably an integer of 0 to 9. The —CH(CH₂)ₘCH— group may preferably have a substituent. The substituent in the —CH(CH₂)ₘCH— group is preferably a reactive functional group, more preferably a functional group having, as a group or a part of a group, an alcohol group, epoxy group, acetal group, orthoester group, ester group, carbonyl group, carboxyl group, anhydrous carboxylic acid group, amide group, imidate group, amino group, imino group, aziridine group, diazo group, azide group, amidyl group, guanidyl group, hydrazyl group, hydrazone group, alkoxyamino group, oxime group, carbonate group, carbamate group, sulfhydryl group, ether group, imide group, thioester group, thioamide group, isothiocyano group, thioether group, disulfide group, halogen group, isocyano group, isocyanate group, oxazirine group, diaziridine group, sulfonyl group, sulfone group, sulfoxide group, sulfonimide group, seleno group, silyl group, boryl group, stannyl group, phosphine group, phosphine oxide group, phosphate group, phosphoric acid ester group, phosphoric acid amide group, methylene group, alkenyl group, or alkynyl group, still more preferably a functional group having, as a group or a part of a group, an alcohol group, epoxy group, ester group, carbonyl-containing group, carboxyl group, anhydrous carboxylic acid group, amide group, amino group, azide group, hydrazone group, oxime group, carbonate group, carbamate group, sulfhydryl group, maleimide group, thioester group, thioamide group, isothiocyano group, thioether group, isocyano group, isocyanate group, oxazirine group, azide group, methanesulfonyl group, p-toluenesulfonyl group, methylene group, alkenyl group, or alkynyl group, or a combination thereof.

In the ABNO derivative in the present invention, in cases where A, B, C, and D each represent CH₂, from the viewpoint of securing a linking group to the desired molecule, at least one of E₁ and E₂ is preferably CR1R2 (wherein at least one of R1 and R2 is a reactive functional group), C═O, C═S, C═NR5, NR5, or SiR6R7, or X and Y preferably together form a —CH(CH₂)ₘCH— group wherein at least one hydrogen on the —CH(CH₂)ₘCH— group is substituted by a reactive functional group. In the ABNO derivative in the present invention, in cases where each of E₁ and E₂ is CH₂, at least one of A, B, C, and D is preferably CHR1.

In another mode, in cases where A, B, C, and D each represent CH₂, and one of the groups E₁ and E₂ represents CH₂, an oxygen atom, or a sulfur atom, the other of the groups E₁ and E₂ preferably represents C═O, C═NH, C═NOH, NH, CHR5, or NR, or C═O, C═NH, C═NOH, C═NOR5, NH, CHR, or NR5.

In another preferred mode, in the ABNO derivative in the present invention, A, B, C, and D each represent CH₂; and one of E₁ and E₂ represents CH₂ or an oxygen atom, and the other represents C═O, CHNH₂, CH(CO)NH₂, or C═NOH. Further, in the above other preferred mode, one of E₁ and E2 preferably represents CH₂. In the above other preferred mode, the ABNO derivative in the present invention can be represented by the following structure.

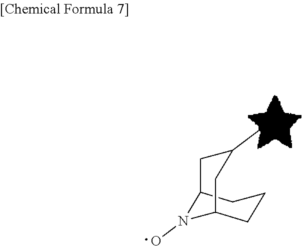

Depending on the desired molecule to be bound, a structure formed by modification of this structure may be used as appropriate.
Examples of the structure of the following moiety:

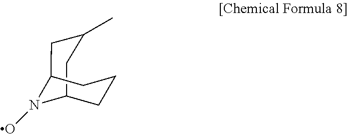

include an oxygen atom (═O), amide, amine, and oxime.

### [Structure of ABNO Derivative]

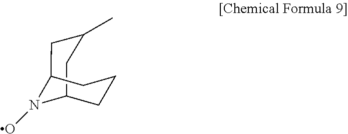

The N-oxy radical group-containing compound or ABNO derivative corresponding to the effective component of the present invention can be obtained by performing chemical modification according to, for example, Sonobe, T., Oisaki, K., Kanai, M. Chem. Sci. 2012, 3, 1572-1576; Lauber, M. B., Stahl, S. S. ACS Catal. 2013, 3, 2612-2616; Hayashi, M., Sasano, Y., Nagasawa, S., Shibuya, M., Iwabuchi, Y. Chem. Pharm. Bull 2011, 59, 1570; Sasano, Y., Nishiyama, T., Tomizawa, M., Shibuya, M., Iwabuchi, Y. Heterocycles 2013, 87, 2109; and, when necessary, according to Greg T. Hermanson “Bioconjugate Techniques, third edition”. As an effective component of the ABNO derivative in the present invention, a commercially available product may be used as long as the structure of Formula (I) is satisfied.

The cross-linking agent of the present invention may contain a solvent, catalyst, buffer, or another known additive in addition to the N-oxy radical group-containing compound or ABNO derivative depending on conditions of the cross-linking reaction and/or the like, or may be constituted only by the radical compound or ABNO derivative. The content of the radical compound or ABNO derivative in the cross-linking agent of the present invention is, for example, 0.1 to 100% by mass. Thus, according to one mode, the ABNO derivative in the present invention is composed of the compound represented by Formula (I).

### Indole-Structure-Containing Molecule

The ABNO derivative in the present invention is capable of site-selectively bonding to an indole structure in an indole-structure-containing molecule. The radical oxygen at an end of the ABNO derivative in the present invention is advantageous for selective bonding to especially the 3-position of the indole structure represented by the following Formula.

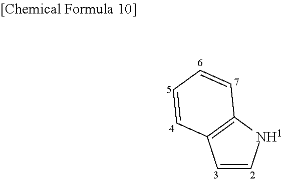

The indole-structure-containing molecule in the present invention may be obtained as a naturally occurring molecule, may be prepared by modification of a naturally occurring molecule, may be produced by synthesis, or may be a commercially available product.

Examples of the indole-structure-containing molecule include peptides, lipids, sugars, nucleic acids, and cells, and linked bodies thereof, and fibers, peptides, proteins, metal complexes, organic dyes, organic electronic materials, and polymer materials. The molecule is not limited as long as it has the above structure. Since the indole structure is a structure contained in the amino acid tryptophan, one mode of the indole-structure-containing molecule Is a molecule containing a tryptophan structure. Representative examples of the molecule containing a tryptophan structure include peptides. A peptide means a molecule in which amino acids are bound to each other via peptide bonds, and includes the so-called polypeptides and proteins. Examples of the proteins include enzymes, membrane proteins, and antibodies, and also protein aggregates. Thus, according to a preferred mode of the present invention, the indole-structure-containing molecule is a peptide containing tryptophan. Since the cross-linking agent of the present invention selectively reacts with an indole structure such as tryptophan present on a protein surface, it is especially advantageous to use a protein containing tryptophan from the viewpoint of forming a conjugate while maintaining the spatial structure and the function of the protein.

According to a preferred mode of the present invention, the Indole structure can be represented by the following Formula (II).

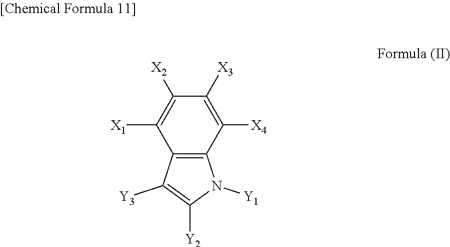

(wherein in the formula,

X₁, X₂, X₃, X₄, Y₂, and Y₃ each independently represent a hydrogen atom; or a functional group capable of substituting a hydrogen atom on an indole ring;

the functional group capable of substituting a hydrogen atom on an Indole ring is optionally a halogen atom; heteroatom group; C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, or C₃-C₃₀ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₇-C₃₀ aralkyloxy group which is optionally substituted, or C₃-C₃₀ cycloalkyloxy group which is optionally substituted; or polyalkyleneoxy group which Is optionally substituted;

at least two of the groups X₁, X₂, X₃, X₄, Y₂, and Y₃ optionally together form a 4- to 10-membered ring; and

Y₁ represents a hydrogen atom, or a functional group capable of substituting a hydrogen atom on nitrogen).

The alkyl group, alkenyl group, alkynyl group, aryl group, heteroaryl group, aralkyl group, or cycloalkyl group represented by each of X₁, X₂, X₃, X₄, Y₂, and Y₃ may be selected from the same groups as those for the alkyl group, alkenyl group, alkynyl group, aryl group, heteroaryl group, aralkyl group, or cycloalkyl group, respectively, represented by each of R1, R2, R3, R4, R6, R7, and R8.

The alkyl group, alkenyl group, alkynyl group, aryl group, heteroaryl group, aralkyl group, or cycloalkyl group constituting part of the alkyloxy group, alkenyloxy group, alkynyloxy group, aryloxy group, heteroaryloxy group, aralkyloxy group, or cycloalkyloxy group, respectively, represented by each of X₁, X₂, X₃, X₄, Y₂, and Y₃ may also be selected from the same groups as those for the alkyl group, alkenyl group, alkynyl group, aryl group, heteroaryl group, aralkyl group, or cycloalkyl group, respectively, in each of R1, R2, R3, R4, R6, R7, and R8.

The 4- to 10-membered ring which at least two of the groups X₁, X₂, X₃, X₄, Y₂, and Y₃ together form is preferably a 4- to 8-membered carbon ring or hetero ring, more preferably a 4- to 6-membered carbon ring or hetero ring. The hetero ring is preferably a pyridyl group, triazole group, or the like.

In Formula (II), X₁, X₂, X₃, and X₄ are each preferably Independently a hydrogen atom; halogen atom; heteroatom; C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, or C₃-C₃₀ cycloalkyl group which is optionally substituted; or C₁-C₃₀ alkyloxy group which is optionally substituted, C₂-C₃₀ alkenyloxy group which is optionally substituted, C₂-C₃₀ alkynyloxy group which Is optionally substituted, C₆-C₃₀ aryloxy group which is optionally substituted, C₄-C₃₀ heteroaryloxy group which is optionally substituted, C₇-C₃₀ aralkyloxy group which is optionally substituted, or C₃-C₃₀ cycloalkyloxy group which is optionally substituted; more preferably a halogen atom; hydroxyl group; or C₁-C₃₀ alkyl group which is optionally substituted or C₁-C₃₀ alkyloxy group which is optionally substituted; still more preferably a hydrogen atom.

In Formula (II), Y₁ is a hydrogen atom or a functional group capable of substituting a hydrogen atom on nitrogen, preferably a hydrogen atom or a protecting group for an amino group. Examples of the protecting group for an amino group include, but are not limited to, the protecting groups described in pp. 494 to 653 in Theodora W. Greene and Peter G. M. Wuts, Protective groups in Organic Chemistry (3rd ed., published by JOHN WILEY & SONS, INC). The protecting group for an amino group is preferably a carbamate-type protecting group such as methoxycarbonyl, ethoxycarbonyl, isopropoxycarbonyl, tert-butoxycarbonyl, allyloxycarbonyl, benzyloxycarbonyl, or phenoxycarbonyl; acyl-type protecting group such as formyl, acetyl, trichioroacetyl, trifluoroacetyl, benzoyl, or p-nitrobenzoyl; or benzyl; more preferably a carbamate-type protecting group such as methoxycarbonyl, ethoxycarbonyl, isopropoxycarbonyl, tert-butoxycarbonyl, allyloxycarbonyl, benzyloxycarbonyl, or phenoxycarbonyl; especially preferably a carbamate-type protecting group (for example, benzyloxycarbonyl). Preferred examples of the protecting group for an amino group in the present invention also include the alkyl group, alkenyl group, alkynyl group, aryl group, heteroaryl group, aralkyl group, or cycloalkyl group in each of R1, R2, R3, R4, R6, R7, and R8. The present invention also includes such modes.

In Formula (II), Y₂ and Y₃ are preferably a combination of a hydrogen atom(s), hydroxyl group(s), and/or —(CH₂)ₒCGF₁F₂. More preferably, Y₁ and Y₂ are hydrogen atoms, and Y₃ is —(CH₂)ₒCGF₁F₂.

In Formula (II), a preferred combination of Y₁, Y₂, and Y₃ is preferably a combination of a hydrogen atom(s), hydroxyl group(s), and/or —(CH₂)ₒCGF₁F₂. More preferably, Y₁ and Y₂ are each a hydrogen atom or a hydroxyl group, and Y₃ is —(CH₂)ₒCGF₁F₂.

In the (CH₂)ₒCGF₁F₂ group in Formula (II), O may be an integer of 0 to 10, preferably 0 to 5, more preferably 0 to 3, still more preferably 0 to 2, still more preferably 1 or 2.

In the (CH₂)ₒCGF₁F₂ group in Formula (II), G may be a hydrogen atom, alkyl group, alkyloxy group, or aryl group. G is preferably a hydrogen atom, alkyl group, alkyloxy group, or aryl group.

In the (CH₂)ₒCGF₁F₂ group in Formula (II), F₁ and F₂ may each independently represent —NHCOZ₁ or —CONHZ₂. Preferably, one of F₁ and F₂ represents —NHCOZ₁, and the other represents —CONHZ₂.

Z₁ and Z₂ each represent a natural product, a synthetic product, or a linked body thereof. Examples of such a natural product or synthetic product Include, but are not limited to, peptides, lipids, sugars, nucleic acids, and cells, and linked bodies thereof. Z₁ and Z₂ may also be other than peptides, lipids, sugars, nucleic acids, or cells, or linked bodies thereof. The present invention also includes such modes.

In cases where a linker constitutes a part of the group in each of Z₁ and Z₂, a known linker may be used as the linker depending on the structure of the bonding molecule and/or the like. The linker is not limited, and may be, for example, a linker described in Greg T. Hermanson, “Bioconjugate Techniques, third edition”.

### Desired Molecule

In the present invention, an Indole-structure-containing molecule can be selectively cross-linked to a desired molecule through an ABNO derivative. Examples of the desired molecule include drugs, toxins, labeling substances (organic dyes, fluorescent proteins, histidine, biotin, and the like), fibers, peptides, proteins, nucleic acids, cells, organic electronic materials, and polymer materials. For example, an antibody-drug conjugate can be prepared by using an antibody as the indole-structure-containing molecule (protein), and using a drug as the desired molecule.

The bonding of the cross-linking agent of the present invention to the desired molecule can be allowed to occur between, for example, a substituent (a group capable of bonding to the desired molecule, represented by at least one of the groups A, B, C, D, E₁, and E₂ in Formula (I)) in the cross-linking agent and a functional group in the desired molecule. For example, the bonding can be allowed to occur with an alkoxyamino group in the desired-molecule side in cases where a carbonyl group is present in the cross-linking-agent side, with an amino group in the desired-molecule side in cases where a carboxyl group is present in the cross-linking-agent side, or with a carboxyl group in the desired-molecule side in cases where an amine is present in the cross-linking-agent side. Thus, the desired molecule in the present invention preferably has an alkoxyamino group, amino group, or carboxyl group.

The desired molecule may be obtained as a naturally occurring molecule, may be prepared by modification of a naturally occurring molecule, may be produced by synthesis, or may be a commercially available product.

### Conjugate

As described above, by the present Invention, a conjugate between an indole-structure-containing molecule and a desired molecule can be provided by site-specific cross-linking of the indole-structure-containing molecule to the desired molecule through an ABNO derivative. Thus, according to one mode of the present invention, a conjugate formed by cross-linking through an ABNO derivative is provided.

In the present invention, from the viewpoint of maintaining the structure and the function of the indole-structure-containing molecule, it is preferred to form the conjugate using an indole-structure-containing molecule similar to a tryptophan-containing peptide such as the one shown in the Formula (II). One preferred mode of the conjugate obtained using the indole-structure-containing molecule shown in Formula (II) is the conjugate shown in the following Formula (III).

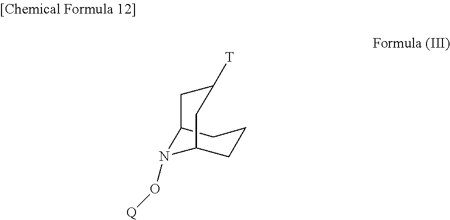

[wherein in the formula,

T is a desired molecule linked to the condensed bicyclic structure in Formula (III);

Q is a group represented by either Formula (IV) or Formula (V):

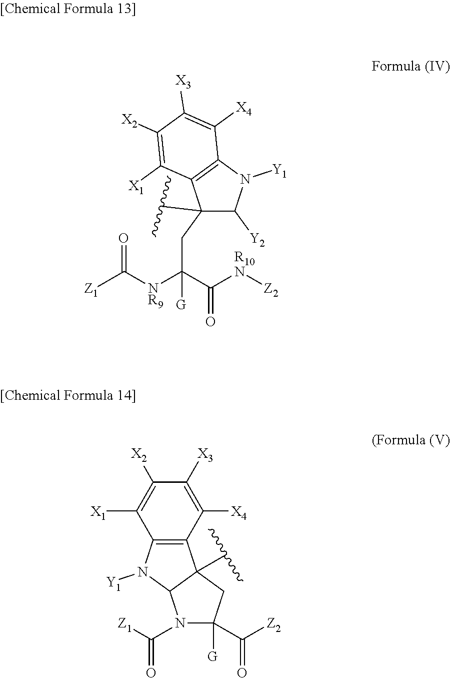

(wherein in the formulae,

X₁, X₂, X₃, X₄, and Y2 are each a hydrogen atom; or a functional group capable of substituting a hydrogen atom on an indole ring;

Y₁ represents a functional group capable of substituting a hydrogen atom on a nitrogen atom;

R9 and R10 are each a hydrogen atom; or a functional group capable of substituting a hydrogen atom on an amide group;

at least two of the groups X₁, X₂, X₃, and X₄ optionally together form a 4- to 10-membered ring;

G represents hydrogen, alkyl group, alkyloxy group, or aryl group; and

Z₁ and Z₂ are each a natural product, a synthetic product, or a linked body thereof)].

In production of the conjugate of Formula (III), a compound in which each of A, B, C, D, and E₂ is CH₂; E₁ is CHR1; and R1 is a reactive functional group; in Formula (I) may be preferably used as the ABNO derivative.

In the conjugate of Formula (III), T may represent a desired molecule linked to the ABNO derivative through a reactive functional group (R5). Preferred examples of the desired molecule in Formula (III) include, as described above, drugs, toxins, labeling substances (organic dyes, fluorescent proteins, and the like), fibers, peptides, proteins, nucleic acids, cells, organic electronic materials, and polymer materials. Q may preferably represent an indole-structure-containing molecule similar to tryptophan (compound of Formula (II)) bound to the terminal radical oxygen of the ABNO derivative.

In cases where the tryptophan-like indole structure is bound to the terminal radical oxygen of the ABNO derivative, a block represented by Formula (IV) or Formula (V) can be formed. The wavy line in each of Formula (IV) and Formula (V) indicates the site bound to the terminal radical oxygen of the ABNO derivative.

In each of Formula (IV) and Formula (V), the specific types of the substituents X₁, X₂, X₃, X₄, Y₁, Y₂, G, Z₁, and Z₂, and their combination may be preferably the same as in Formula (II). In a more preferred mode, in each of Formula (IV) and Formula (V), X₁, X₂, X₃, X₄, and G each represent a hydrogen atom. In a still more preferred mode, in Formula (IV), Y₁ is a protecting group for an amino group, and Y₂ is a hydroxyl group.

### Method for Producing Conjugate

As described above, according to the present invention, a conjugate can be produced by efficiently and specifically cross-linking an indole-structure-containing molecule to a desired molecule using the radical compound or ABNO derivative in the present Invention as a cross-linking agent. Thus, according to another mode of the present invention, a method for producing a conjugate comprising the step of cross-linking an Indole-structure-containing molecule to a desired molecule through the cross-linking agent is provided.

In the method for producing a conjugate of the present Invention, the order of the bonding reactions for the indole-structure-containing molecule, the cross-linking agent, and the desired molecule is not limited as long as formation of the conjugate is not Inhibited. Either the indole-structure-containing molecule or the desired molecule may be first bound to the cross-linking agent. Thus, according to one mode, the method for producing a conjugate includes the step of allowing a desired molecule to act on a cross-linking agent (the radical compound or ABNO derivative), and then allowing the cross-linking agent, to which the desired molecule Is bound, to act on an indole structure in an indole-structure-containing molecule. According to another mode, the method for producing a conjugate includes the step of allowing a cross-linking agent (the radical compound or ABNO derivative) to act on an indole structure in an indole-structure-containing molecule, and then allowing a desired molecule to act on the cross-linking agent bound to the indole-structure-containing molecule.

In the method for producing a conjugate of the present invention, each step for cross-linking may be carried out in, for example, a water solvent, organic solvent, or a mixed solvent thereof. From the viewpoint of simplicity and safety, each step is preferably carried out in a water solvent or an aqueous solvent (a solvent containing water as an indispensable component, and, when necessary, an organic solvent). The fact that the cross-linking agent of the present invention is highly soluble in water and can be used for modification reaction in water is a discovery achieved by the present inventors. In view of the fact that compounds having similar structures are insoluble in water, this discovery was unexpected by those skilled in the art at the time of filing of the present application. Accordingly, the fact that ABNO derivatives can be used for reaction in a water solvent or an aqueous solvent was unexpected by those skilled in the art at the time of filing of the present application.

Examples of the organic solvent that may be used in the production method of the present invention Include nitrlle-based solvents, amide-based solvents, alcohol-based solvents, ether-based solvents, ketone-based solvents, ester-based solvents, hydrocarbon-based solvents, sulfoxide-based solvents, and halogen-based solvents. Specific examples of these solvents include, but are not limited to, methanol, n-hexanol, t-butyl alcohol, ethylene glycol, acetone, methyl ethyl ketone, cyclohexanone, n-hexane, toluene, xylene, diethyl ether, dioxane, ethyl acetate, acetonitrile, methylformamide, dimethylformamide, dimethylacetamide, dimethylsulfoxide, and methylene chloride.

In cases where a biomolecule such as a peptide is used as the Indole-structure-containing molecule in the reaction between the indole-structure-containing molecule and the ABNO derivative, the reaction is especially preferably carried out in a water solvent from the viewpoint of maintaining the spatial structure of the molecule.

In the reaction between the indole-structure-containing molecule and the cross-linking agent, an activating agent/oxidizing agent for generation of active species, such as a nitrite, Brønsted acid, metal catalyst, photocatalyst, peracid, or oxygen may be used. In cases where a nitrite is used for the reaction, it is preferably used in an amount of 0.6 to 3 equivalents with respect to 1 equivalent of the cross-linking agent. In cases where a Brønsted acid is used, it is preferably used in an amount in which the pH of the reaction solution becomes 5 to 6. In cases where a metal catalyst or a photocatalyst is used, it is preferably used in an amount of not more than 1 equivalent as long as it can function as a catalyst. In cases where a peracid is used, it is preferably used in an amount of not less than 1 equivalent. In cases where oxygen is used, it is preferably used at normal pressure. The temperature and the reaction time for the reaction between the indole-structure-containing molecule and the cross-linking agent described above may be appropriately controlled by those skilled in the art depending on, for example, the type of the catalyst and the amount of each reaction component. In cases where a nitrite is used for the reaction, the temperature may be, for example, −10 to 60° C., preferably 20 to 40° C. The reaction time may be, for example, about 1 minute to 24 hours.

In a preferred mode, the reaction between the ABNO derivative and the desired molecule is a cross-linking reaction between a group(s) constituted by at least one of A, B, C, D, E₁, and E₂ capable of linking to the desired molecule [C═O, C═NH, CH₂, NH, CHR5, or NR5 (wherein R5 represents a reactive functional group)] in Formula (I) in the cross-linking agent, and the desired molecule, and may be appropriately carried out by those skilled in the art according to a known cross-linking method taking Into account the type(s) of the group(s) capable of linking to the desired molecule and properties of the desired molecule. Such a known cross-linking method is described in, for example, Greg T. Hermanson, “Bioconjugate Techniques, third edition”, wherein the entire content disclosed in the document is incorporated as a part of the present description by the citation.

### Use and Site-Specific Modification Method

According to another mode of the present invention, use of a compound having an N-oxy radical group and a group capable of bonding to a desired molecule, as a cross-linking agent for an indole-structure-containing molecule and the desired molecule is provided. According to still another preferred mode, use of a compound having an N-oxy radical group and a group capable of bonding to a desired molecule, in production of a conjugate between an indole-structure-containing molecule and the desired molecule is provided. In either of the above modes, the compound is an ABNO derivative.

According to another preferred mode of the present invention, a site-specific modification method for an indole-structure-containing molecule, characterized in that a compound having an N-oxy radical group and a group capable of bonding to a desired molecule, or an ABNO derivative, is reacted with the 3-position of the indole-structure-containing molecule, is provided. According to another preferred mode of the present invention, a site-specific modification method for a protein containing an indole-structure-containing molecule, characterized in that a compound having an N-oxy radical group and a group capable of bonding to a desired molecule, or an ABNO derivative, is reacted with the 3-position of the indole-structure-containing molecule, is provided. In the above method, the spatial structure of the protein is substantially maintained. The maintenance of the spatial structure can be confirmed by the method described in the following examples. The modes of the use and the site-specific modification method described above can be carried out by those skilled in the art according to the method for producing a conjugate described above.

### EXAMPLES

The present invention is described below by way of Examples. However, the present invention is not limited to the following Examples. Unless otherwise specified, the units and the measurement methods in the present description are as defined in Japanese Industrial Standards (JIS).

### Example 1: Method for Producing 3-((2-(2-(2-(2-Azidoethoxy)ethoxy)ethoxy)ethoxy)imino)-9-aza bicyclo[3.3.1]nonan-9-yl Benzoate

According to the following procedure (Scheme 1), 3-((2-(2-(2-(2-azidoethoxy)ethoxy)ethoxy)ethoxy)imino)-9-aza bicyclo[3.3.1]nonan-9-yl benzoate was prepared.

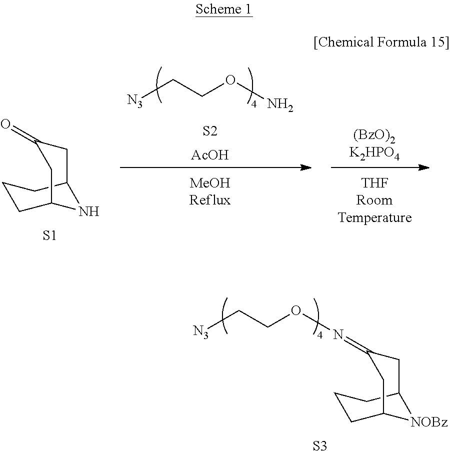

Under an argon atmosphere, S1 (9-azabicyclo[3.3.1]nonan-3-one) (598.5 mg, 4.3 mmol), S2 (O-(2-(2-(2-(2-azidoethoxy)ethoxy)ethoxy)ethyl)hydroxyamine) (1.1 g, 4.7 mmol), acetic acid (269.1 μl, 4.7 mmol), and 21.5 ml of methanol were placed in a flask, and mixed together. The mixture was stirred under reflux for 21 hours. The mixture was then concentrated under reduced pressure. To the residual solution, 21.5 ml of THF (tetrahydrofuran) was added, and then (BzO)₂ (benzodiazepine, 75% by weight, 1.5 g, 4.7 mmol) and K?HPO₄ (973.7 mg, 5.6 mmol) were added thereto under an argon atmosphere. The mixture was stirred at room temperature for 25.5 hours, and then H₂O was added thereto. The resulting mixture was extracted with ethyl acetate, and the combined organic phase was washed with H₂O and brine, followed by drying over Na₂SO₄. Na₂SO₄ was separated by filtration, and the filtrate was concentrated under reduced pressure. The resulting residue was purified by flash chromatography (elution with hexane/ethyl acetate=1/1 to 1/2) using a silica gel column (manufactured by Merck, Product No. 1.09385.9025), to obtain 3-((2-(2-(2-(2-azidoethoxy)ethoxy)ethoxy)ethoxy)imino)-9-aza bicyclo[3.3.1]nonan-9-yl benzoate as a colorless liquid (1.1 g; yield, 53%). The analysis data for 3-((2-(2-(2-(2-azidoethoxy)ethoxy)ethoxy)ethoxy)imino)-9-aza bicyclo[3.3.1]nonan-9-yl benzoate were as follows.

¹H NMR (CDCl₃): δ=1.42 (d, J=13.4, 1H), 1.74-1.88 (m, 3H), 2.24 (d, J=17.9, 2H), 2.33 (d, J=16.4 Hz, 1H), 2.63 (dd, 3=16.5, 5.1 Hz, 1H), 2.99 (dd, J=16.5, 5.1 Hz, 1H), 3.11 (d, J=16.4 Hz, 1H), 3.37 (t, 3=4.7 Hz, 2H), 3.67 (s, 10H), 3.74 (t, J=4.7 Hz, 2H), 3.80 (s, 1H), 3.87 (s, 1H), 4.22 (t, J=4.7 Hz, 2H), 7.44 (dd, J=7.4, 7.0 Hz, 2H), 7.57 (t, J=7.4 Hz, 1H), 7.99 (d, J=7.0 Hz, 2H); ¹³C NMR (CDCl₃): δ=164.4, 156.9, 133.0, 129.7, 129.3, 128.5, 72.8, 70.74, 70.68, 70.0, 69.9, 57.1, 56.3, 50.8, 32.3, 31.8, 29.9, 25.0, 15.7; IR (KBr): 2932, 2106, 1740, 1450, 1247, 1062, 711 cm⁻¹; LRMS (ESI): m/z 498 [M+Na]⁺; HRMS (ESI): m/z calcd for C₂₃H₃₃N₅O₆Na [M+Na]⁺ 498.2324, found 498.2348.

### Example 2: Preparation of Functional Linker (1) (Preparation of ABNO-fluorescein Methyl Ester Linker)

An ABNO-fluorescein methyl ester (FL) linker was prepared according to the following procedure (Scheme 2).

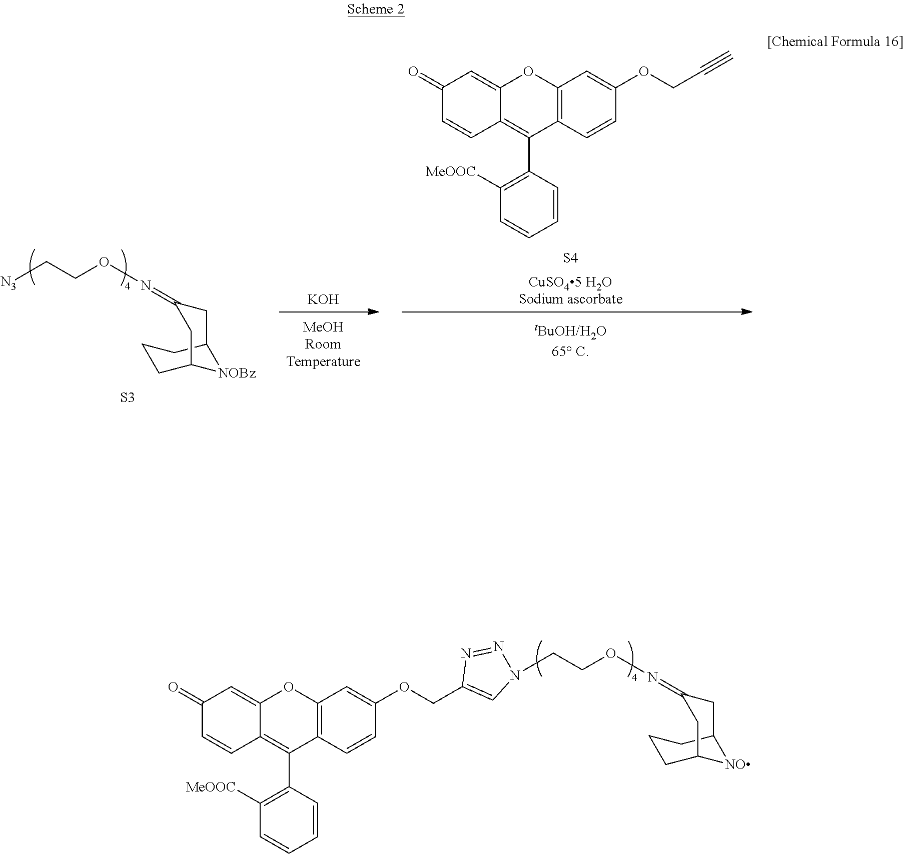

In a flask, 3-((2-(2-(2-(2-azidoethoxy)ethoxy)ethoxy)ethoxy)imino)-9-aza bicyclo[3.3.1]nonan-9-yl benzoate obtained by the method of Example 1 (47.6 mg, 0.1 mmol), 10% KOH solution in methanol (0.2 ml), and methanol (3.3 ml) were placed, and then mixed together. The mixture was stirred at room temperature for 5 hours. The mixture was then concentrated under reduced pressure. The resulting residue was filtered, and washed with CH₂Cl₂, followed by concentrating the filtrate under reduced pressure. To the residue in the flask, S4 (methyl 2-(3-oxo-6-(prop-2-yn-1-yloxy)-3H-xanthen-9-yl) benzoate) (38.4 mg, 0.1 mmol), CuSO₄.5H₂O (1.3 mg, 0.005 mmol), sodium ascorbate (9.9 mg, 0.05 mmol), tert-butyl alcohol (1.5 ml), and H₂O (0.5 ml) were added under an argon atmosphere. The mixture was stirred at 65° C. for 12.5 hours. The mixture was then concentrated under reduced pressure. The resulting residue was purified by flash chromatography (elution with CH₂Cl₂/methanol=20/1 to 10/1) using a silica gel column (manufactured by Merck, Product No. 1.09385.9025), to obtain an ABNO-fluorescein methyl ester (FL) linker as an orange solid (53.5 mg; yield, 71%). The analysis data for the ABNO-FL linker were as follows.

IR (KBr): 3398, 2924, 1723, 1642, 1597, 1509, 1106 cm⁻¹; LRMS (ESI): m/z 778 [M+Na]⁺; HRMS (ESI): m/z calcd for C₄₀H₄₅N₅O₁₀Na [M+Na]⁺ 778.3059, found 778.3055.

### Example 3: Preparation of Functional Linker (2) Preparation of ABNO-Biotin Linker)

An ABNO-biotin linker was prepared according to the following procedure (Scheme 3).

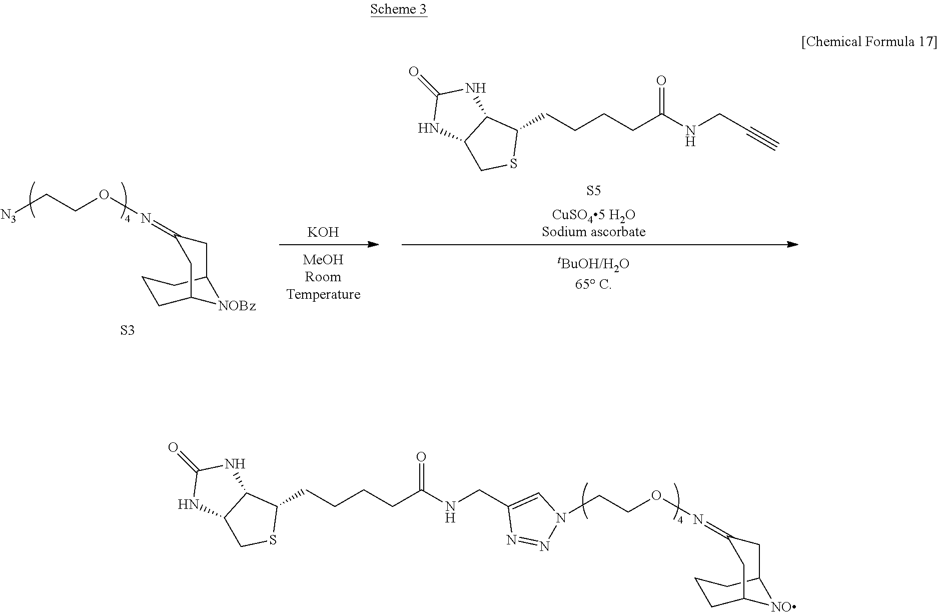

In a flask, 3-((2-(2-(2-(2-azidoethoxy)ethoxy)ethoxy)ethoxy)imino)-9-aza bicyclo[3.3.1]nonan-9-yl benzoate obtained by the method of Example 1 (95.1 mg, 0.2 mmol), 10% KOH solution in methanol (0.4 ml), and methanol (6.7 ml) were placed, and then mixed together. The mixture was stirred at room temperature for 19 hours. The mixture was then concentrated under reduced pressure. To the residue in the flask, S5 (5-((3aS,4S,6aR)-2-oxohexahydro-1H-thieno[3,4-d]imidazol-4-yl)-N-(prop-2-yn-1-yl)pentanamide) (56.3 mg, 0.2 mmol), CuSO₄.5H₂O (2.5 mg, 0.01 mmol), sodium ascorbate (19.8 mg, 0.1 mmol), tert-butyl alcohol (1 ml), and H₂O (1 ml) were added under an argon atmosphere. The mixture was stirred at 65° C. for 17.5 hours. The mixture was then concentrated under reduced pressure. The resulting residue was purified by flash chromatography (elution with CH₂Cl₂/methanol=5/1) using a silica gel column (manufactured by Merck, Product No. 1.09385.9025), to obtain an ABNO-biotin linker as a yellow solid (56.1 mg; yield, 43%). The analysis data for the obtained ABNO-biotin linker were as follows.

IR (KBr): 3289, 2931, 2604, 2496, 1697, 1462, 1106 cm⁻¹; LRMS (ESI): m/z 675 [M+Na]⁺; HRMS (ESI): m/z calcd for C₂₉H₄₈N₈O₇SNa [M+Na]⁺ 675.3259, found 675.3276.

### Example 4: Preparation of Functional Linker (3) (Preparation of ABNO-Anticancer Drug SN38 Linker)

An ABNO-anticancer drug SN38 linker was prepared according to the following procedure (Scheme 4).

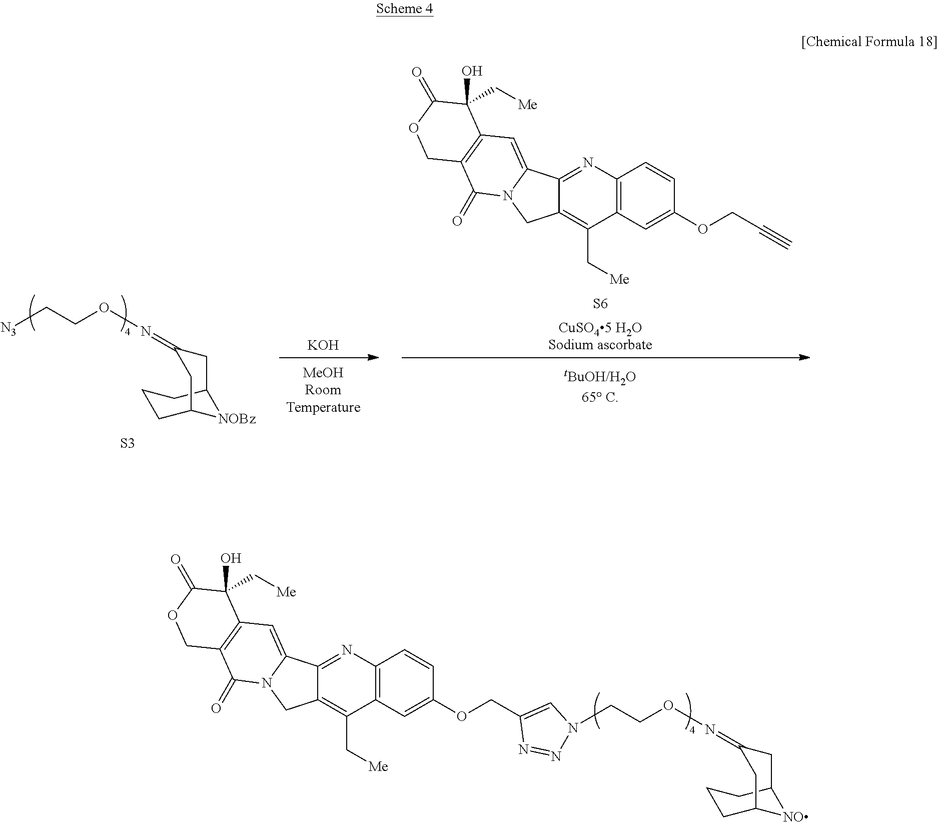

In a flask, 3-((2-(2-(2-(2-azidoethoxy)ethoxy)ethoxy)ethoxy)imino)-9-aza bicyclo[3.3.1]nonan-9-yl benzoate obtained by the method of Example 1 (38 mg, 0.08 mmol), 10% KOH solution in methanol (0.16 ml), and methanol (2.7 ml) were placed, and then mixed together. The mixture was stirred at room temperature for 8 hours. The mixture was then concentrated under reduced pressure. The resulting residue was filtered, and washed with CH₂Cl₂, followed by concentrating the filtrate under reduced pressure. To the residue in the flask, S6 ((S)-4,11-diethyl-4-hydroxy-9-(prop-2-yn-1-yloxy)-1,12-dihydro-14H-pyrano[3,4′:6,7]indolizino[1,2-b]quinoline-3,14(4H)-dione) (34.4 mg, 0.08 mmol), CuSO₄.5H₂O (1 mg, 0.004 mmol), sodium ascorbate (7.9 mg, 0.04 mmol), tert-butyl alcohol (0.4 ml), and H₂O (0.4 ml) were added under an argon atmosphere. The mixture was stirred at 65° C. for 13.5 hours. The mixture was then concentrated under reduced pressure. The resulting residue was purified by flash chromatography (elution with hexane/ethyl acetate=1/5 to CH₂Cl₂/methanol/triethyl amine=10/1/1 liter) using a silica gel column (manufactured by Merck, Product No. 1.09385.9025), to obtain an ABNO-anticancer drug SN38 linker as a yellow solid (48.8 mg; yield, 76%). The analysis data for the ABNO-anticancer drug SN38 linker were as follows.

IR (KBr): 3386, 2934, 2495, 2102, 1748, 1655, 1595, 1108, 830 cm⁻¹; LRMS (ESI): m/z 824 [M+Na]⁺; HRMS (ESI): m/z calcd for C₄₁H₅₁N₇O₁₀Na [M+Na]⁺ 824.3590, found 824.3588.

### Example 5: Bonding of ABNO Derivative to Biologically Active Peptide in Water Solvent

Formation of conjugates between an ABNO derivative and each of the biologically active peptides shown below in a water solvent was confirmed according to the following procedure (Scheme 5).

Neuromedin B (manufactured by Peptide Institute, Inc., Product No. 4152)

LH-RH (gonadotropin-releasing hormone, manufactured by Peptide Institute, Inc., Product No. 4013-v)

Kisspeptin-10 (manufactured by Peptide Institute, Inc., Product No. 4389-v)

DSIP (delta sleep inducing peptide, manufactured by Peptide Institute, Inc., Product No. 4054-v)

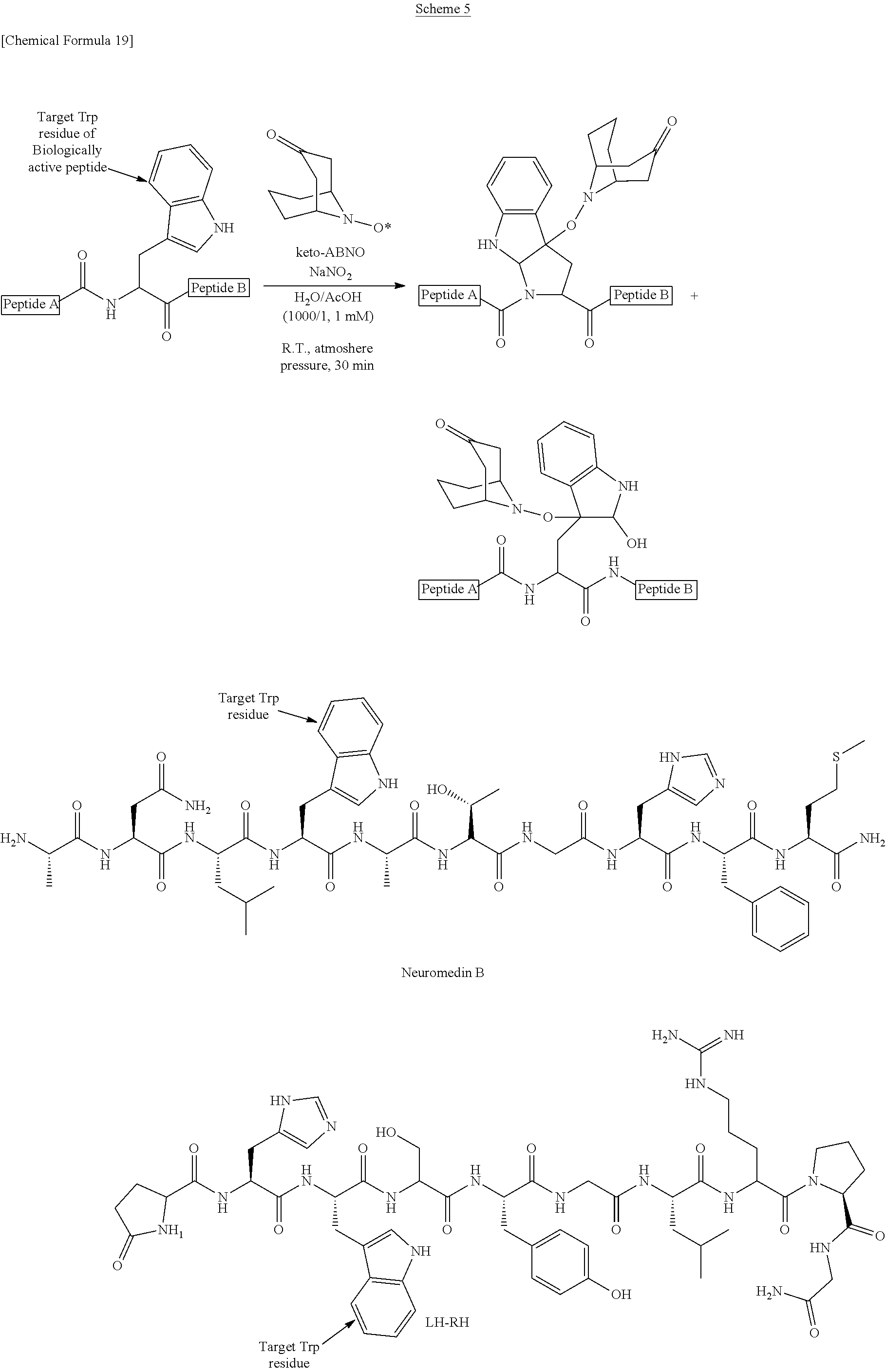

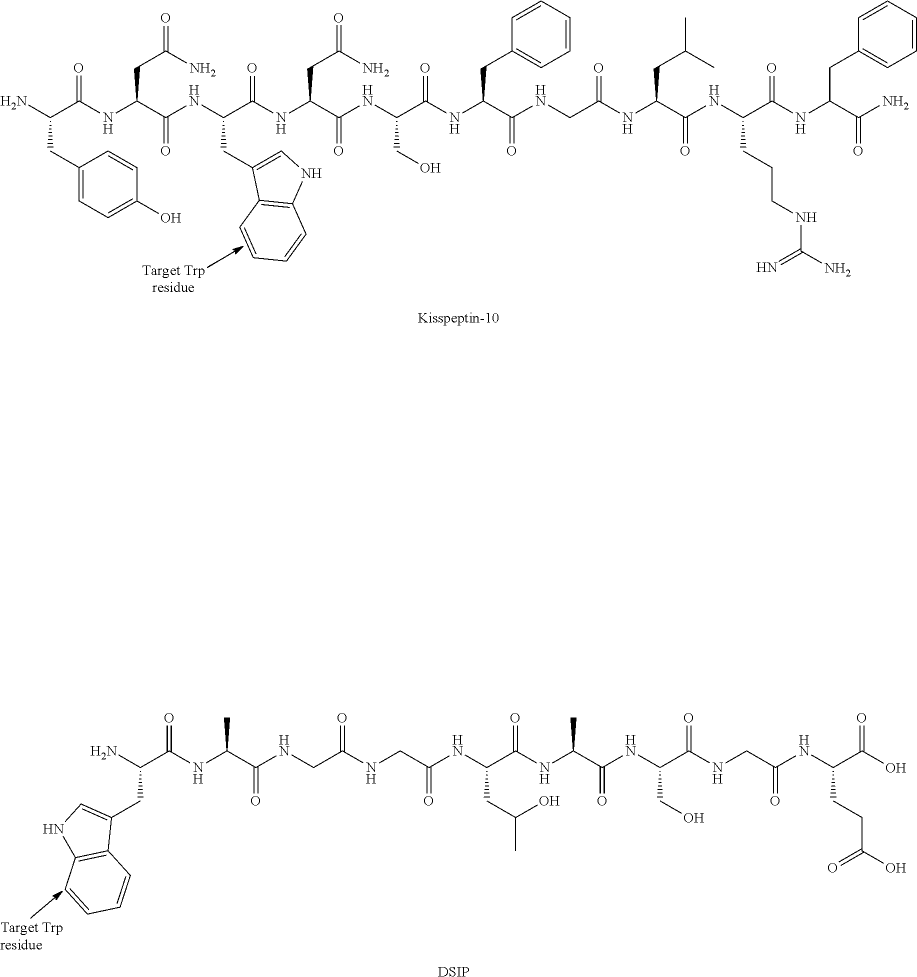 [SMILES: CC(C)C[C@H](NC(=O)CNC(=O)[C@H](Cc1ccccc1)NC(=O)[C@H](CO)NC(=O)[C@H](CC(N)=O)NC(=O)[C@H](Cc1c[nH]c2ccccc12)NC(=O)[C@H](CC(N)=O)NC(=O)[C@@H](N)Cc1ccc(O)cc1)C(=O)N[C@@H](CCCNC(=N)N)C(=O)NC(Cc1ccccc1)C(N)=O ; CC(O)C[C@H](NC(=O)CNC(=O)CNC(=O)[C@H](C)NC(=O)[C@@H](N)Cc1c[nH]c2ccccc12)C(=O)N[C@@H](C)C(=O)N[C@@H](CO)C(=O)NCC(=O)N[C@@H](CCC(=O)O)C(=O)O ; "DSIP"]

One of the above biologically active peptides (0.2 μmol), the ABNO derivative keto-ABNO (0.2 μmol, 20 μl of 10 mM aqueous solution), NaNO₂ (0.12 μmol, 12 μl of 10 mM aqueous solution), AcOH (0.2 μl), and H₂O (168 μl) were placed in an Eppendorf tube, and then mixed together. The mixture was stirred at room temperature in the atmosphere for 30 minutes, and the reaction was quenched with PBS buffer (pH 7.4), to obtain a conjugate between keto-ABNO and each biologically active peptide.

During the above reaction, the reaction was monitored using an LC/MS spectrophotometer. LC analysis was carried out under the following conditions.

Apparatus: Agilent 6200 equipped with 1260 infinity (manufactured by Agilent Technologies)

Column: C18 reverse-phase column (4.6 mm×150 mm; YMC-Triart C18 column, manufactured by YMC Co., Ltd.)

Temperature: room temperature (25° C.)

Mobile phase: 0% to 100% linear gradient of acetonitrile containing 0.1% TFA, 40 minutes

Flow rate: 1 ml/minute

Injection volume: 10 μl

Detection wavelengths: 230 nm and 250 nm (absorption wavelengths for amide bonds)

From the ratio between the total peak area of all detected peaks and the total peak area of the biologically active peptide obtained by the LC analysis, the HPLC yield of the conjugate between keto-ABNO and each biologically active peptide was calculated. The HPLC yield of the conjugate between keto-ABNO and each biologically active peptide was 84% for the Neuromedin B conjugate, 91% for the LH-RH conjugate, 83% for the Kisspeptin-10 conjugate, and 84% for the DSIP conjugate.

As a result of NMR analysis and X-ray crystallography of the conjugate between keto-ABNO and each biologically active peptide produced, it was found that the two kinds of conjugates shown in Scheme 5 are produced for each biologically active peptide.

From these results, formation of the conjugates between the ABNO derivative and the biologically active peptides in the water solvent was shown by the present invention.

### Example 6: Bonding of ABNO Derivative to Protein in Water Solvent (1)

Formation of a conjugate between an ABNO derivative and a protein (lysozyme) In a water solvent was confirmed according to the following procedure.

Lysozyme (manufactured by Sigma-Aldrich, Product No. L-4919, 1.1 mg, 0.08 μmol), the ABNO derivative keto-ABNO (0.08 μmol, 8 μl of 10 mM aqueous solution), NaNO₂ (0.05 μmol, 0.16 μl of 0.3 mM aqueous solution), AcOH (0.088 μl), and H₂O (84.6 μl) were placed in an Eppendorf tube, and then mixed together. The mixture was stirred at room temperature in the atmosphere for 30 minutes, and the reaction was quenched by addition of PBS buffer (pH 7.4), to obtain a keto-ABNO-lysozyme conjugate.

During the above reaction, the reaction was monitored using an LC/MS spectrophotometer and by mass spectrometry with deconvolution. LC analysis was carried out under the following conditions.

Apparatus: Agilent 6200 equipped with 1260 infinity (manufactured by Agilent Technologies)

Column: C18 reverse-phase column (4.6 mm×150 mm; YMC-Triart C18 column, manufactured by YMC Co., Ltd.)

Temperature: room temperature (25° C.)

Mobile phase: 0% to 100% linear gradient of acetonitrile containing 0.1% TFA, 40 minutes

Flow rate: 1 ml/minute

Injection volume: 10 μl

Detection wavelength: 230 nm (wavelength for amide bonds)

From the result of the LC analysis, formation of the conjugate between the ABNO derivative and lysozyme in the water solvent was shown by the present invention.

### Example 7: Bonding of ABNO Derivative to Protein in Waater Solvent (2)

Formation of a conjugate between an ABNO derivative and a protein (lysozyme) in a water solvent was confirmed by X-ray crystallography according to the following procedure.

Lysozyme (manufactured by Sigma-Aldrich, Product No. L-4919, 11.4 mg, 0.8 μmol), the ABNO derivative keto-ABNO (0.8 μmol, 80 μl of 10 mM aqueous solution), NaNO₂ (2.4 μmol, 8 μl of 0.3 M aqueous solution), AcOH (4.4 μl), and water (835.6 μl) were placed in an Eppendorf tube, and then mixed together. The mixture was stirred at room temperature in the atmosphere for 30 minutes. The mixture was then concentrated to 100 mg/ml using an Amicon centrifugal filter to provide a test solution. After mixing 1 μl of the test solution with the same amount of a crystallization reagent (8M NaCl, 0.05M AcOH (pH 4.5)), the obtained droplet was subjected to the hanging drop vapor diffusion method at room temperature to perform neutralization with the crystallization reagent (200 μl), to obtain crystals. The obtained crystals were charged into a low-temperature proteIn crystallization tool (CryoLoop, manufactured by Hampton Research Co.), and instantly cooled under a 95-K nitrogen gas flow. Subsequently, X-ray diffraction data were collected using a protein single-crystal structure analyzer (RIGAKU Micro 7 HFM-AXIS7) under the following conditions.

Measurement temperature: 95 K

Amplitude: 0.5°

Exposure time per frame: 2 minutes

Crystal-detector distance: 150 mm

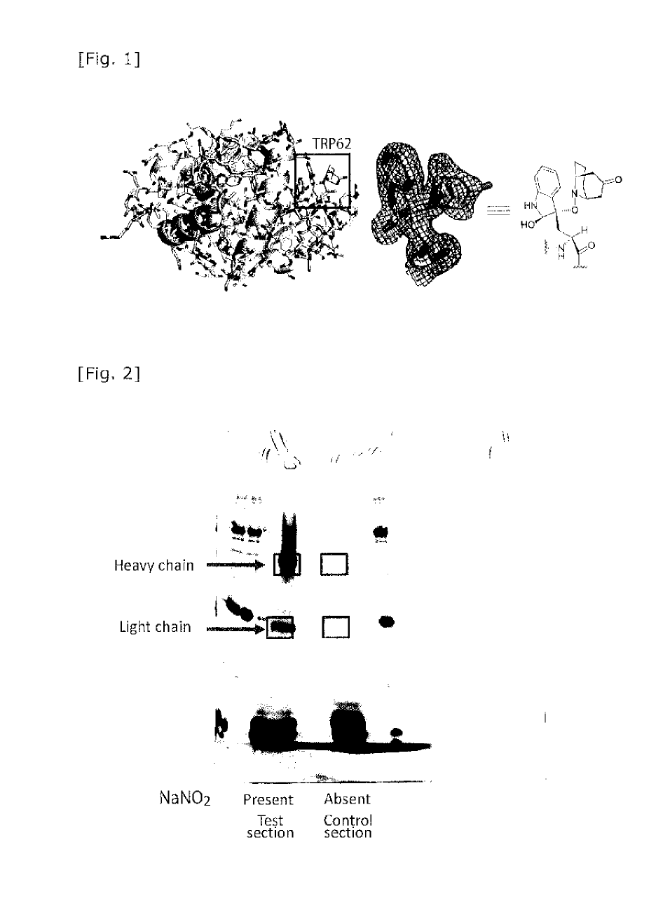

Each diffraction image was subjected to integration and calculation using the structure analysis programs iMosflm, and Scala in CCP4i. Using, as a search model, the structure of lysozyme deposited in the Protein Data Bank of RCSB with the ID “2LYZ”, the structure of the keto-ABNO-lysozyme conjugate in the test solution was determined by the molecular substitution method using the program Molrep. The obtained model was modified and refined using the programs Coot and Refmac. For final validation of the structure, Ramachandran analysis was carried out using the MolProbity program. The structure of the keto-ABNO-lysozyme conjugate obtained as a result of the analysis is shown in FIG. 1.

As Is evident from FIG. 1, formation of the conjugate between the ABNO derivative and lysozyme in the water solvent was shown also by X-ray crystallography by the present invention.

### Example 8: Bonding of ABNO Derivative to Antibody in Water Solvent (1)

Formation of conjugates between an ABNO derivative and an anti-amyloid β antibody (anti-Aβ₁₋₁₆ antibody) 6E10 in a water solvent was confirmed according to the following procedure.

An anti-amyloid β antibody 6E10 (manufactured by BioLegend, Product No. 803001) (66.7 μmol, 10 μl of 1 mg/ml aqueous solution), a keto-ABNO-fluorescein methyl ester (FL) linker obtained by the method of Example 2 (1.3 nmol, 10 μl of 133.6 μM aqueous solution), NaNO₂ (0.8 nmol, 0.08 μl of 10 mM aqueous solution), and AcOH (0.1 μl) were placed in an Eppendorf tube, and then mixed together. The mixture was stirred at room temperature in the atmosphere for 30 minutes, to obtain a reaction solution in a test group.

On the other hand, a reaction solution in a control group was obtained in the same manner as in the above method except that the same amount of water was added Instead of the aqueous NaNO₂ solution.

The reaction solution in each of the test group and the control group was mixed with 5×SDS loading buffer (0.25 M Tris (pH 6.8), 10% SDS, 30% glycerol, and 0.05% bromophenol blue), and 5% 2-mercaptoethanol, and the resulting mixture was boiled for 5 minutes. The mixture was then subjected to electrophoresis using Extra PAGE One Precast Gel (manufactured by Nacalai Tesque, Inc.) together with SDS running buffer (25 mM Tris, 0.19 M glycine, and 0.1% SDS). The gel after the electrophoresis was stained with Coomassie Brilliant Blue. The result is shown in FIG. 2.

The molecular weights of the stained bands were assumed with a molecular weight marker Precision Plus Protein™ Dual Color Standards (manufactured by Bio-Rad Laboratories, Inc.). Based on the molecular weights of the heavy chain and the light chain of 6E10, and the theoretical values of the molecular weights of keto-ABNO and fluorescein methyl ester, it was assumed that fluorescein methyl ester (FL) formed a conjugate with each of the heavy chain and the light chain of 6E10 through keto-ABNO in the test group. From this result, formation of the conjugates between the ABNO derivative and the anti-amyloid β antibody (anti-Aβ₁₋₁₆ antibody) 6E10 in the water solvent was shown by the present invention.

### Example 9: Bonding of ABNO Derivative to Antibody in Water Solvent (2)

Formation of a conjugate between a functional molecule and an anti-Aβ antibody 6E10 through an ABNO derivative in a water solvent, and maintenance of the bonding capacity of the anti-Aβ antibody 6E10 in the formed conjugate to amyloid β, were confirmed by a dot blot assay.

Twenty microliters of a solution of 1.4 mM or 7 mM O-acyl-isoamyloid β₁₋₄₂ (manufactured by Peptide Institute, Inc.) in TFA (0.1%) was diluted with the same amount of phosphate buffer (0.2 M, pH 7.4). Immediately after the dilution, 30 μl of 100 mM phosphate buffer (pH 7.4) was added thereto to allow rearrangement of the acyl group from O- to N-, to prepare 0.4 mM or 2 mM Aβ₁₋₄₂.

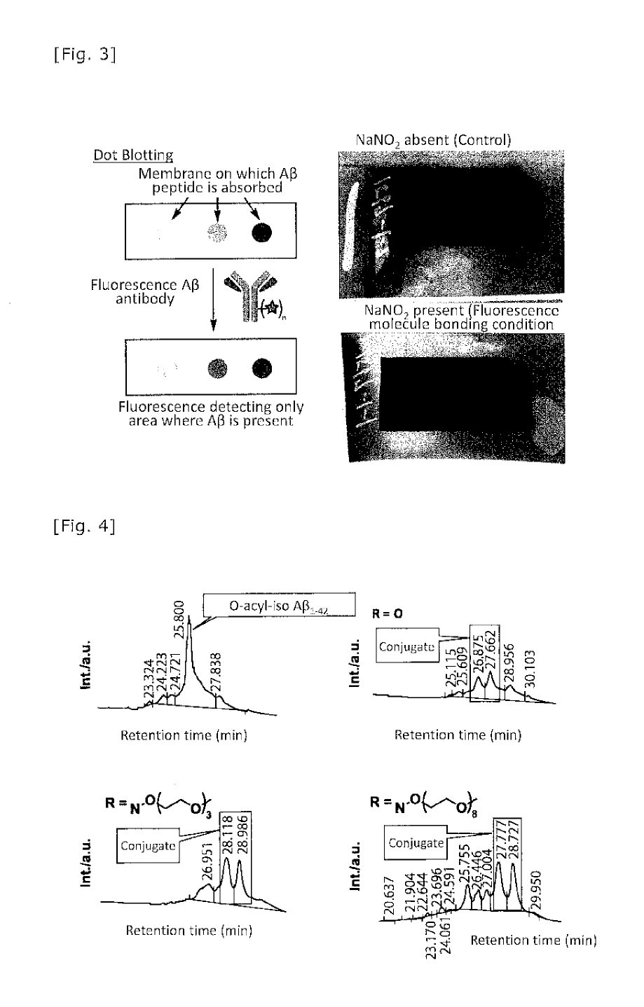

The obtained 0.4 mM or 2 mM Aβ₁₋₄₂ was attached once or three times to a PVDF blotting membrane (manufactured by GE Healthcare Life Sciences, Co.) for spot formation, and then the PVDF membrane was dried. Subsequently, the PVDF membrane was blocked at room temperature for 1 hour with TBS-T (1M Tris-HCl 2%, Tween 20 0.1%, pH 8.5) supplemented with 3% BSA. The membrane was then washed three times (for 10 minutes each) with TBS-T, and incubated in TBS-T supplemented with 3% BSA at room temperature for 1 hour together with 20 μl of a reaction solution for each of a test group and a control group prepared by the same method as in Example 8. After the Incubation, the PVDF membrane was washed three times (for 10 minutes each) with TBS-T. Using the laser scanner Typhoon FLA 9000 (manufactured by GE Healthcare Japan, Co.), the fluorescence intensity of each spot on the PVDF membrane was measured. The result is shown in FIG. 3.

As is evident from FIG. 3, a conjugate was formed between fluorescein methyl ester (FL) and the antibody 6E10 through keto-ABNO in the test group. In the 6E10 that formed the conjugate with keto-ABNO-FL, the bonding capacity to amyloid 1 was maintained. Thus, formation of the conjugate between FL and the anti-Aβ antibody 6E10 through the ABNO derivative in the water solvent, and maintenance of the bonding capacity of the anti-Aβ antibody 6E10 in the formed conjugate to amyloid β, were shown by the present invention.

### Example 10: Bonding of ABNO Derivative to Indole Structure in Organic Solvent (1)

Formation of a conjugate between an ABNO derivative and an indole structure in an organic solvent was confirmed according to the following procedure (Scheme 6).

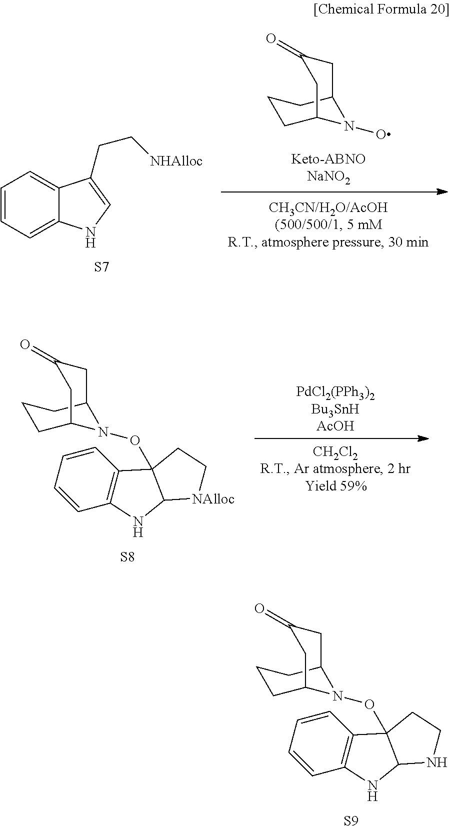

S7 (N^{ω}-Alloc-tryptamine) (0.4 mmol, 97.6 mg), which has an indole structure, the ABNO derivative keto-ABNO (0.6 mmol, 92.5 mg), NaNO₂ (0.2 mmol, 16.5 mg), CH₃CN (40 ml), AcOH (80 μl), and H₂O (40 ml) were placed in a flask, and then mixed together. The mixture was stirred at room temperature in the atmosphere for 30 minutes. The resulting mixture was extracted with ethyl acetate, and the combined organic phase was washed with H₂O and brine, followed by drying over Na₂SO₄. Na₂SO₄ was separated by filtration, and the filtrate was concentrated under reduced pressure. The resulting residue was purified by flash chromatography (elution with hexane/ethyl acetate=1/2) using a silica gel column (manufactured by Merck, Product No. 1.09385.9025), to obtain S8 compound as a colorless liquid (123.5 mg). The analysis data for S8 after removal of the Alloc group were as follows.

IR (KBr): 3363, 2946, 1699, 1613, 1471, 1407, 1196, 747, 633 cm⁻¹; LRMS (ESI): m/z 420 [M+Na]⁺; HRMS (ESI): m/z calcd for C₂₂H₂₇NO₄Na [M+Na]⁺ 420.1894, found 420.1888.

Bu₃SnH (0.3 mmol, 91.5 μl) was added to a mixture of S8 (0.3 mmol, 123.5 mg), PdCl₂ (PPh₃)₂ (16 μmol, 11.2 mg), AcOH (0.7 mmol, 42.4 μl), and CH₂Cl₂ (6.2 ml). The mixture was stirred at room temperature for 2 hours, and then an aqueous NaHCO₃ solution was added thereto. The resulting mixture was extracted with CH₂Cl₂/MeOH=20/1, and the combined organic phase was washed with H₂O and brine, followed by drying over Na₂SO₄. Na₂SO₄ was separated by filtration, and the filtrate was concentrated under reduced pressure. The resulting residue was purified by flash chromatography (elution with CH₂Cl₂/MeOH=10/1) using a silica gel column (manufactured by Merck, Product No. 1.09385.9025), to obtain S9 compound as a white solid (78.2 mg; yield, 59%). The analysis data for S9 were as follows.

¹H NMR (CDCl₃): δ=1.22-1.30 (m, 2H), 1.62-1.74 (m, 2H), 1.87-1.91 (m, 3H), 2.09 (d, J=16.2 Hz, 2H), 2.16-2.21 (m, 1H), 2.32-2.39 (m, 1H), 2.81-2.88 (m, 1H), 3.00-3.13 (m, 3H), 3.55 (s, 1H), 3.64 (s, 1H), 4.33 (brs, 1H), 5.16 (s, 1H), 6.64 (d, J=7.4 Hz, 1H), 6.76 (dd, J=7.2 Hz, 1H), 7.13 (dd, J=7.4, 7.2 Hz, 1H), 7.32 (d, J=7.2 Hz, 1H); ¹³C NMR (CDCl₃): δ=211.3, 151.5, 129.8, 129.4, 125.1, 118.9, 110.1, 98.9, 81.0, 60.2, 59.9, 45.6, 41.01, 40.97, 39.5, 31.81, 31.75, 15.4; IR (KBr): 3347, 3054, 2944, 1693, 1607, 1469, 1100, 1024, 743 cm⁻¹; LRMS (ESI): m/z 336 [M+Na]⁺; HRMS (ESI): m/z calcd for C₁₈H₂₃N₃O₂Na [M+Na]⁺ 336.1683, found 336.1693.

From these results, formation of a conjugate between the ABNO derivative and the indole structure in the organic solvent was shown by the present invention.

### Example 11: Bonding of ABNO Derivative to Indole Structure in Organic Solvent (2)

Formation of a conjugate between an ABNO derivative and an indole structure in an organic solvent was confirmed by X-ray crystallography according to the following procedure (Scheme 7).

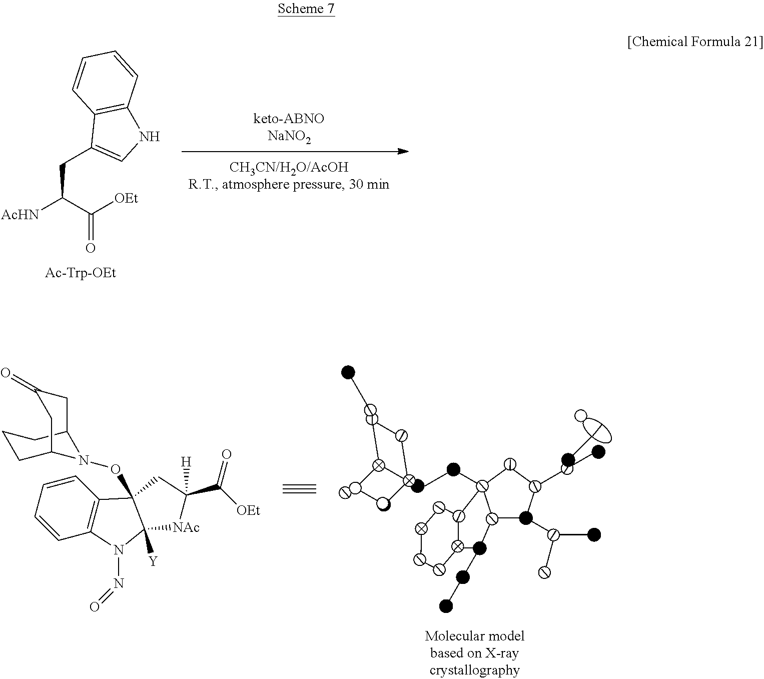

N^{α}-acetyltryptophan ethyl ester (0.2 mmol, 54.9 mg), which has an indole structure, the ABNO derivative keto-ABNO (0.2 mmol, 30.8 mg), NaNO₂ (0.3 mmol, 20.7 mg), CH₃CN (10 ml), AcOH (2.3 ml), and H₂O (10 ml) were placed in a recovery flask, and then mixed together. The mixture was stirred at room temperature in the atmosphere for 30 minutes. The resulting mixture was extracted with ethyl acetate, and the combined organic phase was washed with H₂O and brine, followed by drying over Na₂SO₄. Na₂SO₄ was separated by filtration, and the filtrate was concentrated under reduced pressure. The resulting residue was purified by flash chromatography (elution with dichloromethane/methanol=40/1) using a silica gel column (manufactured by Merck, Product No. 1.09385.9025).

According to the same methods as in Example 7, collection of X-ray diffraction data and X-ray crystallography were carried out. From the result of the “molecular model based on X-ray crystallography”, formation of a conjugate between the ABNO derivative and the indole structure in the organic solvent was shown by the present invention.

### Example 12: Bonding of ABNO Derivative to Biologically Active Peptide in Organic Solvent

Formation of a conjugate between an ABNO derivative and the biologically active peptide Neuromedin B in an organic solvent was confirmed according to the following procedure (Scheme 8).

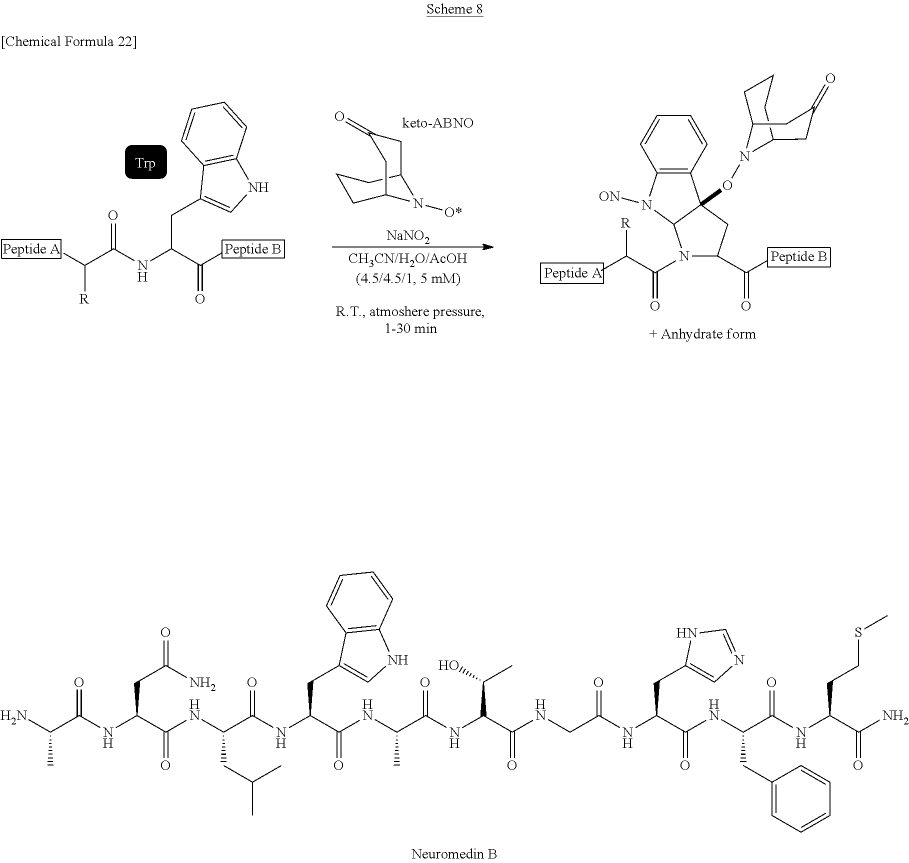

Neuromedin B (0.2 μmol), the ABNO derivative keto-ABNO (0.2 μmol, 20 μl of 10 mM solution in CH₃CN), NaNO₂ (0.12 μmol, 12 μl of 10 mM aqueous solution), AcOH (0.2 μl), and H₂O (168 μl) were placed in an Eppendorf tube, and then mixed together. The mixture was stirred at room temperature in the atmosphere for 30 minutes, and the reaction was quenched with PBS buffer (pH 7.4), to obtain a conjugate between Neuromedin B and keto-ABNO.

During the above reaction, the reaction was monitored using an LC/MS spectrophotometer. LC analysis was carried out under the following conditions.

Apparatus: Agilent 6200 equipped with 1260 infinity (manufactured by Agilent Technologies)

Column: C18 reverse-phase column (4.6 mm×150 mm; YMC-Triart C18 column, manufactured by YMC Co., Ltd.)

Temperature: room temperature (25° C.)

Mobile phase: 0% to 100% linear gradient of acetonitrile containing 0.1% TFA, 40 minutes

Flow rate: 1 ml/minute

Injection volume: 10 μl

Detection wavelengths: 230 nm and 250 nm (absorption wavelengths for amlde bonds)

From the ratio between the total peak area of all detected peaks and the total peak area of Neuromedin B obtained by the LC analysis, the HPLC yield of the conjugate between keto-ABNO and Neuromedin B was calculated. The HPLC yield of the conjugate between keto-ABNO and Neuromedin B was 70%.

From this result, formation of the conjugate between the ABNO derivative and the biologically active peptide in the organic solvent was shown by the present invention.

### Example 13: Bonding of ABNO Derivatives to Protein in Organic Solvent

Formation of conjugates between ABNO derivatives and O-acyl-isoamyloid β₁₋₄₂ (O-acyl-isoAβ₁₋₄₂-Trp) having a tryptophan residue attached to the C-terminus, through the tryptophan residue at the C-terminus, in an organic solvent was confirmed according to the following procedure (Scheme 9).

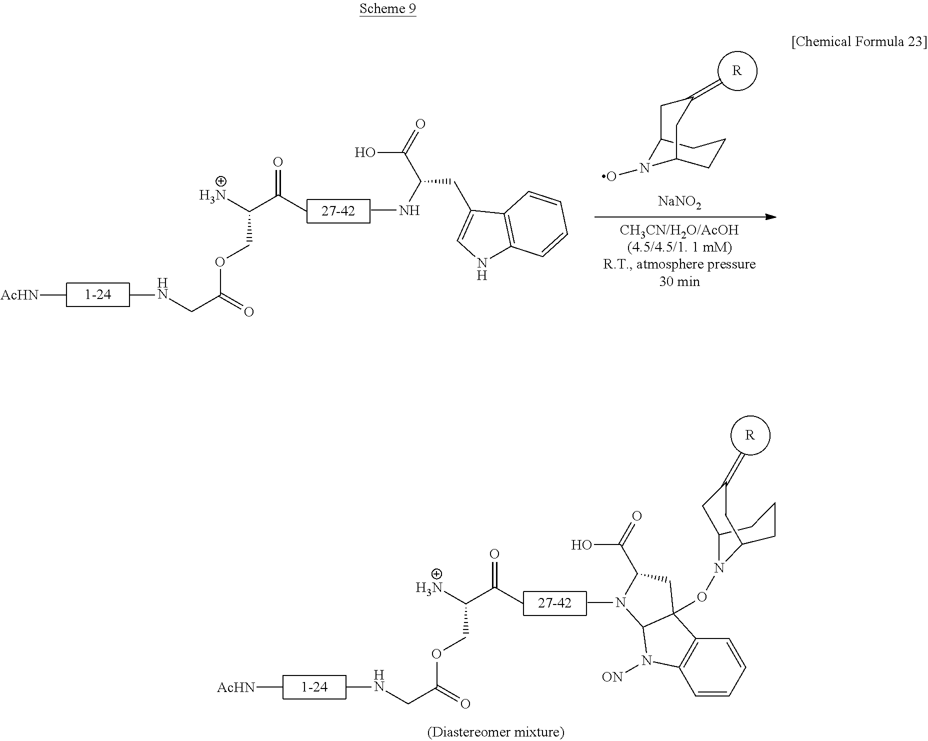

In the above scheme, R represents 0, —NO(CH₂CH₂O)₃CH₃, or —NO(CH₂CH₂O)₈CH₃.

In an Eppendorf tube, 0.2 μmol of O-acyl-isoAβ₁₋₄₂-Trp, 0.3 μmol of one of the above ABNO derivatives (30 μl of 10 mM solution in CH₃CN), 0.18 μmol of NaNO₂ (18 μl of 10 mM aqueous solution), CH₃CN (70 μl), AcOH (0.3 μl), and H₂O (82 μl) were placed, and then mixed together. The mixture was stirred at room temperature in the atmosphere for 30 minutes, and the reaction was quenched with PBS buffer (pH 7.4), to obtain a conjugate between O-acyl-isoAβ₁₋₄₂-Trp and the ABNO derivative.

HPLC analysis was carried out for each obtained conjugate under the following conditions.

Apparatus: HPLC system (manufactured by JASCO Corporation; detector, UV-2075; pump, PU-2080; degassing apparatus, DG-2080-54; mixer, MX-2080-32)

Column: C18 reverse-phase column (4.6 mm×150 mm; YMC-Triart C18 column, manufactured by YMC Co., Ltd.)

Temperature: room temperature (25° C.)

Mobile phase: 0% to 100% linear gradient of acetonitrile containing 0.1% TFA, 40 minutes

Flow rate: 1 ml/minute

Injection volume: 100 μl

Detection wavelength: 230 nm (absorption wavelength for amide bonds)

The HPLC charts for O-acyl-isoAβ₁₋₄₂-Trp and each conjugate are shown in FIG. 4.

From the results in FIG. 4, formation of the conjugate between each ABNO derivative and O-acyl-isoAβ₁₋₄₂-Trp through the tryptophan residue attached to the C-terminus of the O-acyl-isoAβ₁₋₄₂-Trp, in the organic solvent was shown by the present invention.

### Example 14: Study of Influence on Higher-order Structure of Protein

Influence of formation of a conjugate by bonding of keto-ABNO to a protein on the higher-order structure of the protein in the conjugate was studied according to the following procedure.

A keto-ABNO-lysozyme conjugate prepared by the same method as in Example 6 was dissolved in water, and the concentration was adjusted to 20 μM to provide an aqueous solution for a test group. On the other hand, lysozyme alone was dissolved in water, and the concentration was adjusted to 20 μM to provide an aqueous solution for a control group.

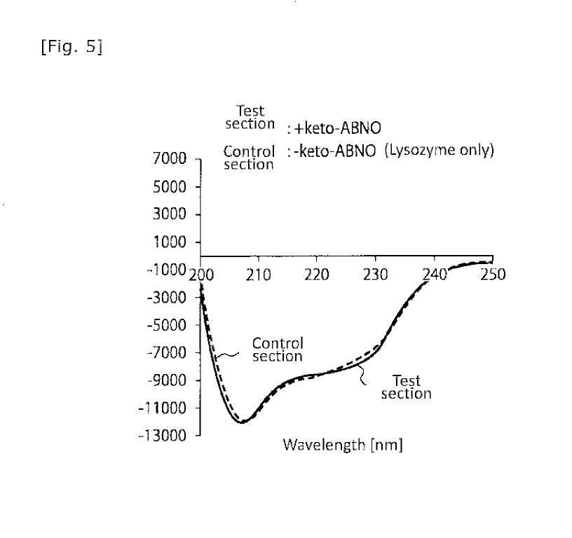

Using a circular dichroism (CD) dispersion meter Model 202SF (manufactured by AVIV Biomedical, Inc.), the CD spectrum of the aqueous solution in each of the test group and the control group was measured under the following conditions to perform analysis of the higher-order structure of the lysozyme in each aqueous solution. The results are shown in FIG. 5.

Measurement temperature: room temperature (25° C.)

Measurement wavelength: 200 to 250 nm

Data acquisition interval:

Cell length: 1 mm (quartz cell)

Scanning rate: 12 nm/minute (continuous)

Band width: 1 nm

Sensitivity: standard (100 mdeg)

Number of scans: 1

As is evident from FIG. 5, the waveform of the CD spectrum was almost the same between the test group and the control group. It was therefore shown that the higher-order structure of lysozyme forming the conjugate with keto-ABNO is almost the same as the higher-order structure of lysozyme alone. Thus, it was shown that the higher-order structure of lysozyme is not influenced by formation of the conjugate with keto-ABNO.

### Example 15: Comparison with Cysteine Modification Method (1)

From the viewpoint of influence on the higher-order structure of a protein and production of by-products, the method of the present invention and a cysteine modification method (maleimide conjugate addition) were compared according to the following procedure.

A keto-ABNO-lysozyme conjugate prepared by the same method as in Example 6 was used as a test group.

On the other hand, lysozyme bound to phenylmaleimide through a cysteine residue (phenylmaleimide-lysozyme conjugate) was prepared according to the following procedure to provide a control group.

Lysozyme (manufactured by Sigma-Aldrich, Product No. L-4919) (0.08 μmol, 1.1 mg), TCEP (tris(2-carboxyethyl)phosphine hydrochloride) (manufactured by Wako Pure Chemical Industries, Ltd., Product No. 10014) (0.8 μmol, 0.2 mg), and PBS buffer (46.4 μl, pH 7.4) were placed in an Eppendorf tube, and then mixed together. The mixture was stirred at 37° C. for 2 hours, and then N-phenylmaleimide (0.08 μmol, 46.4 μl of 1.7 mM solution in CH₃CN) was added thereto, followed by stirring the resulting mixture at 37° C. for 2 hours, to prepare a phenylmaleimide-lysozyme conjugate.

During the above reaction, the reaction was monitored using an LC/MS spectrophotometer and by mass spectrometry with deconvolution. LC analysis was carried out under the following conditions.

Apparatus: Agilent 6200 equipped with 1260 Infinity (manufactured by Agilent Technologies)

Column: C18 reverse-phase column (4.6 mm×150 mm; YMC-Triart C18 column, manufactured by YMC Co., Ltd.)

Temperature: room temperature (25° C.)

Mobile phase: 0% to 100% linear gradient of acetonitrile containing 0.1% TFA, 40 minutes

Flow rate: 1 ml/minute

Injection volume: 10 μl

Detection wavelength: 230 nm (wavelength for amide bonds)

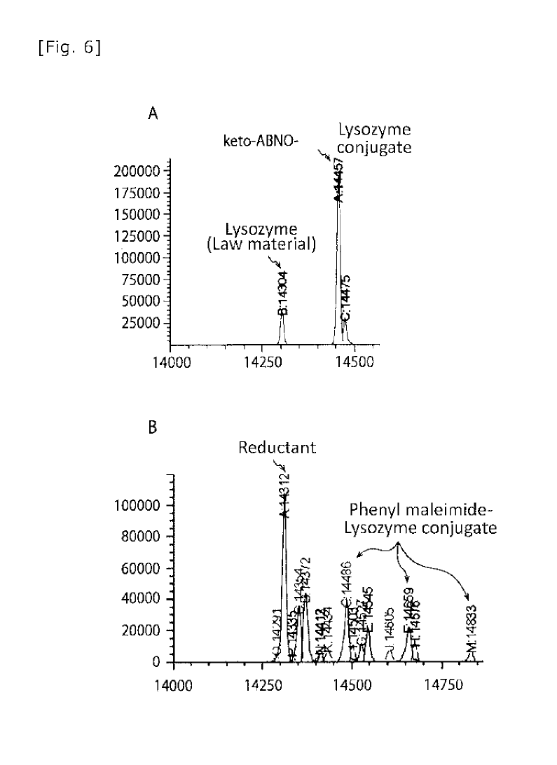

The results of LC analysis of the test group and the control group are shown in FIG. 6A and FIG. 6B, respectively.

In FIG. 6A, the peak shown as “A:14475” indicates a keto-ABNO-lysozyme conjugate, and the peak shown as “B: 14304” indicates lysozyme as a raw material substance.

In FIG. 6B, the peaks shown as “C:14486”, “F:14659”, and “M:14833” indicate different phenylmaleimide-lysozyme conjugates. The peak shown as “A:14312” indicates an Intermediate (reduced form) In a state where a disulfide bond between cysteine residues is reduced in lysozyme as the raw material substance.

From the result in FIG. 6A, it was shown that a keto-ABNO-lysozyme conjugate is formed almost without production of by-products in the test group. On the other hand, from the result in FIG. 6B, it was shown that a plurality of kinds of phenylmaleimide-lysozyme conjugates are formed, and a large number of by-products are produced, in the control group.

### Example 16: Comparison with Cysteine Modification Method (2)

From the viewpoint of influence on the higher-order structure of a protein, the method of the present invention and a cystelne modification method (maleimide conjugate addition) were compared according to the following procedure.

A keto-ABNO-lysozyme conjugate prepared by the method described in Example 6 was used as a test group.

On the other hand, control group A was prepared in the same manner as in the method described in Example 6 except that the same amount of water was added instead of the aqueous NaNO₂ solution. As control group B, lysozyme alone (20 μM) was used. As control group C, a phenylmaleimide-lysozyme conjugate prepared by the method described in Example 15 was used.

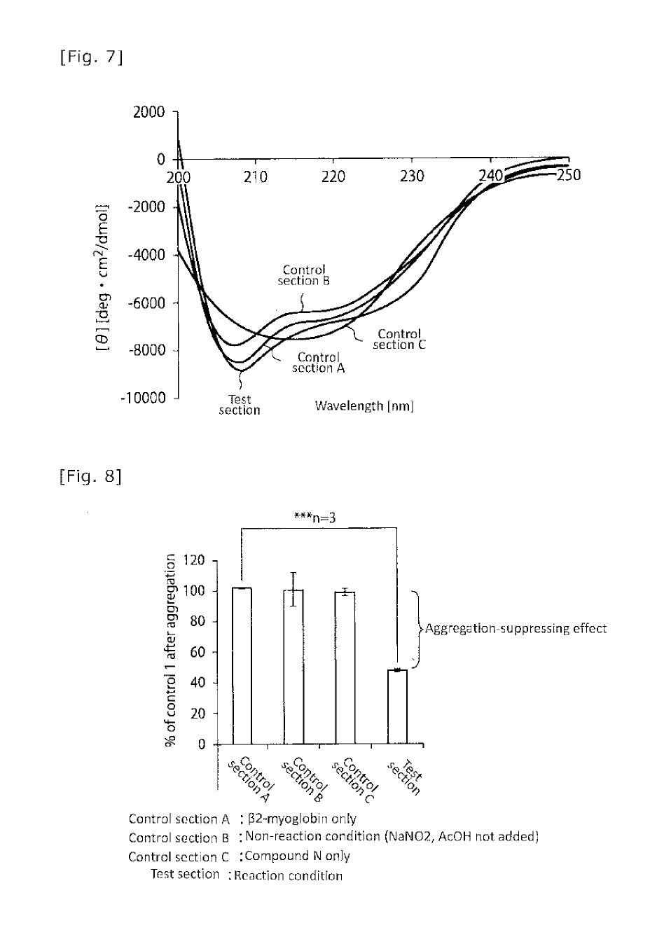

Using a circular dichroism (CD) dispersion meter Model 202SF (manufactured by AVIV Biomedical, Inc.), the CD spectrum was measured for each of the test group and the control groups under the following conditions. The results are shown in FIG. 7.

Measurement temperature: room temperature (25° C.)

Measurement wavelength: 200 to 250 nm

Data acquisition interval:

Cell length: 1 mm (quartz cell)

Scanning rate: 12 nm/mInute (continuous)

Band width: 1 nm

Sensitivity: standard (100 mdeg)

Number of scans: 1

As is evident from FIG. 7, the waveform of the CD spectrum in the test group was almost the same as those of control groups A and B. It was therefore shown that the higher-order structure of lysozyme is maintained in the keto-ABNO-lysozyme conjugate in the test group.

On the other hand, in control group C, the waveform of the CD spectrum was largely different from that of either control group A or B, indicating that the higher-order structure of lysozyme has changed.

### Example 17: Functional Modification of Protein

It was confirmed that formation of a conjugate between keto-ABNO and a protein causes functional modification of the protein. More specifically, a decrease in the aggregability of β2-microglobulin, which is a protein having aggregability, due to formation of a conjugate by bonding of keto-ABNO to a tryptophan residue in the β2-microglobulin, was confirmed by the following procedure.

β2-Microglobulin (2 nmol, 11.5 μl of 174.7 μM aqueous solution), keto-ABNO (2 nmol, 11.5 μl of 174.7 μM aqueous solution), NaNO₂ (1.2 nmol, 0.2 μl of 6 mM aqueous solution), and 2% AcOH (0.11 μl) were placed in an Eppendorf tube, and then mixed together. The mixture was stirred at room temperature in the atmosphere for 30 minutes, and the resulting reaction product was provided as the test group.

On the other hand, as control group A, β2-microglobulin alone (174.7 μM) was used. As control group B, a reaction product obtained in the same manner as the reaction product in the test group except that the aqueous NaNO₂ solution and AcOH were not added was used. As control group C, the compound shown in the following formula alone was used (174.7 μM).

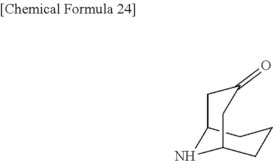

To the reaction product or the single substance in each of the test group and the control groups (10 μl), 5 μl of PBS buffer (100 mM, pH 7.4) and 1.5 μl of 1 M aqueous HCl solution were added. The resulting mixture was stirred at 37° C. for 22 hours, and then 2.25 μl of PBS buffer (100 mM, pH 7.4) and 0.75 μl of 1M aqueous NaOH solution were added thereto, to prepare an aggregate of the reaction product or the single substance in each of the test group and the control groups.

To each of the reaction product or the single substance before the aggregation process (10 μl), and the aggregate of the reaction product or the single substance (10 μl), 5 μM thioflavin-T solution (prepared by adding 4 μl of 500 μM thioflavin-T (manufactured by Sigma-Aldrich) to glycine-NaOH buffer (50 mM, 396 μl, pH 8.5)) was added to provide a sample. The fluorescence intensity of each obtained sample (410 μl) at an emission wavelength of 480 nm was measured at room temperature using an apparatus (Product No. RF-5300PC, manufactured by Shimadzu Corporation) at an excitation wavelength of 440 nm. After the aggregation process, the fluorescence value for each of the test group and the control groups was calculated as a relative percentage with respect to the fluorescence value for control group A (β2-microglobulin alone), which was taken as 100%, to calculate the aggregation-suppressing effect. The results are shown in FIG. 8.

As Is evident from FIG. 8, aggregation of β2-microglobulin was significantly suppressed in the test group. In contrast, aggregation of β2-microglobulin was hardly suppressed in any of control groups A to C.

1: A cross-linking agent suitable for cross-linking an indole-structure-containing molecule to a desired molecule, the cross-linking agent comprising a compound having an N-oxy radical group and a group capable of bonding to the desired molecule.

2: The cross-linking agent according to claim 1, wherein the N-oxy radical group is a dialkylaminooxy radical group.

3: The cross-linking agent according to claim 1, comprising as an effective component an ABNO derivative represented by Formula (I): 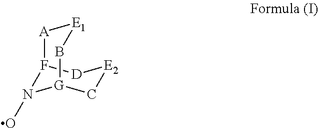
wherein in the formula,
at least one of the groups A, B, C, D, E₁, and E₂ is a group capable of bonding to the desired molecule;
A, B, C, D, E₁, and E₂ each independently represent CR1R2; C═CR3R4; C═O; C═S;
C═NR5; NR5; SiR6R7; an oxygen atom; or a heteroatom other than a nitrogen atom, silicon atom, or oxygen atom;
F and G each represent CR8 or a nitrogen atom;
R1, R2, R3, R4, R5, R6, R7, and R8 each independently represent a hydrogen atom; halogen atom; heteroatom group; C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, or C₃-C₃₀ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₇-C₃₀ aralkyloxy group which is optionally substituted, or C₃-C₃₀ cycloalkyloxy group which is optionally substituted; polyalkyleneoxy group which is optionally substituted; or reactive functional group;
with the proviso that R5 is not a halogen atom; and
E₁ and E₂ optionally together form a —CH(CH₂)ₘCH— group which is optionally substituted, wherein m represents an integer of 0 to 12.

4: The cross-linking agent according to claim 3, wherein
A, B, C, and D each represent CH₂; and
one of E₁ and E₂ represents CH₂, an oxygen atom, or a sulfur atom, and the other represents CR1R2, C═CR3R4, C═O, C═S, C═NR5, NR5, or SiR6R7; or
E₁ and E₂ together form a —CH(CH₂)ₘCH— group which is optionally substituted.

5: The cross-linking agent according to claim 3, wherein the reactive functional group is a functional group having, as a group or a part of a group, an alcohol group, epoxy group, acetal group, orthoester group, ester group, carbonyl group, carboxyl group, anhydrous carboxylic acid group, amide group, imidate group, amino group, imino group, aziridine group, diazo group, azide group, amidyl group, guanidyl group, hydrazyl group, hydrazone group, alkoxyamino group, oxime group, carbonate group, carbamate group, sulfhydryl group, ether group, imide group, thioester group, thioamide group, isothiocyano group, thioether group, disulfide group, halogen group, isocyano group, isocyanate group, oxazirine group, diaziridine group, sulfonyl group, sulfone group, sulfoxide group, sulfonimide group, seleno group, silyl group, boryl group, stannyl group, phosphine group, phosphine oxide group, phosphate group, phosphoric acid ester group, phosphoric acid amide group, methylene group, alkenyl group, or alkynyl group, or a combination thereof.

6: The cross-linking agent according to claim 3, wherein
A, B, C, and D each represent CH₂; and
one of E₁ and E₂ represents CH₂ or an oxygen atom, and the other represents C═O, CHNH₂, CH(CO)NH₂, or C═NOH.

7: The cross-linking agent according to claim 3, wherein one of E₁ and E₂ represents CH₂, and the other represents C═O, CHNH₂, CH(CO)NH₂, or C═NOH.

8: The cross-linking agent according to claim 3, which is suitable for bonding the desired molecule to the indole structure in the indole-structure-containing molecule.

9: The cross-linking agent according to claim 8, wherein the indole structure is represented by Formula (II): 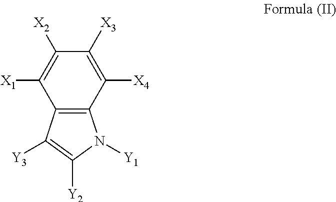
wherein in the formula,
X₁, X₂, X₃, X₄, Y₂, and Y₃ each independently represent a hydrogen atom; or a functional group capable of substituting a hydrogen atom on an indole ring;
the functional group capable of substituting a hydrogen atom on an indole ring is optionally a halogen atom; heteroatom group; C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, or C₃-C₃₀ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₇-C₃₀ aralkyloxy group which is optionally substituted, or C₃-C₃₀ cycloalkyloxy group which is optionally substituted; or polyalkyleneoxy group which is optionally substituted;
at least two of the groups X₁, X₂, X₃, X₄, Y₂, and Y₃ optionally together form a 4- to 10-membered ring; and
Y₁ represents a hydrogen atom, or a functional group capable of substituting a hydrogen atom on nitrogen.

10: The cross-linking agent according to claim 9, wherein
X₁, X₂, X₃, X₄, Y₁, and Y₂ each represent a hydrogen atom;
Y₃ represents —(CH₂)ₒCGF₁F₂;
O represents an integer of 0 to 10;
G represents hydrogen, alkyl group, alkyloxy group, or aryl group;
F₁ and F₂ each independently represent —NR9COZ₁ or —CONR10Z₂;
R9 and R10 each independently represent a hydrogen atom; or a functional group capable of substituting a hydrogen atom on an amide group;
the functional group capable of substituting a hydrogen atom on an amide group is optionally a C₁-C₃₀ alkyl group which is optionally substituted, C₂-C₃₀ alkenyl group which is optionally substituted, C₂-C₃₀ alkynyl group which is optionally substituted, C₆-C₃₀ aryl group which is optionally substituted, C₄-C₃₀ heteroaryl group which is optionally substituted, C₇-C₃₀ aralkyl group which is optionally substituted, or C₃-C₃₀ cycloalkyl group which is optionally substituted; C₁-C₃₀ alkyloxy group which is optionally substituted, C₂-C₃₀ alkenyloxy group which is optionally substituted, C₂-C₃₀ alkynyloxy group which is optionally substituted, C₆-C₃₀ aryloxy group which is optionally substituted, C₄-C₃₀ heteroaryloxy group which is optionally substituted, C₇-C₃₀ aralkyloxy group which is optionally substituted, or C₃-C₃₀ cycloalkyloxy group which is optionally substituted; or polyalkyleneoxy group which is optionally substituted; and
Z₁ and Z₂ are each a natural product, a synthetic product, or a linked body thereof.

11: The cross-linking agent according to claim 1, wherein the indole-structure-containing molecule is a peptide containing tryptophan.

12: The cross-linking agent according to claim 1, wherein the desired molecule is a drug, toxin, labeling substance, fiber, peptide, protein, nucleic acid, cell, organic electronic material, or polymer material.

13: A conjugate cross-linked through the compound recited in claim 1.

14: The conjugate according to claim 13, which is a conjugate formed by bonding of a desired molecule to an indole structure in an indole-structure-containing molecule, wherein the indole structure is cross-linked to the desired molecule through an ABNO derivative.

15: The conjugate according to claim 13, comprising the structure of Formula (III): 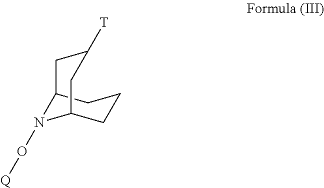
wherein in the formula,
T is the desired molecule linked to the condensed bicyclic structure in Formula (III);
Q is a group represented by either Formula (IV) or Formula (V): 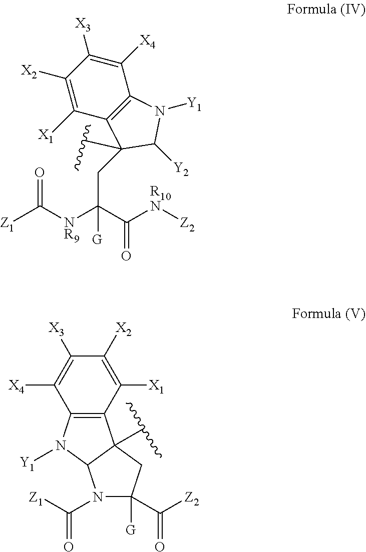
wherein in the formulae,
X₁, X₂, X₃, X₄, and Y₂ are each a hydrogen atom; or a functional group capable of substituting a hydrogen atom on an indole ring;
Y₁ represents a functional group capable of substituting a hydrogen atom on a nitrogen atom;
R9 and R10 are each a hydrogen atom; or a capable group capable of substituting a hydrogen atom on an amide group;
at least two of the groups X₁, X₂, X₃, and X₄ optionally together form a 4- to 10-membered ring;
G represents hydrogen, alkyl group, alkyloxy group, or aryl group; and
Z₁ and Z₂ are each a natural product, a synthetic product, or a linked body thereof.

16: The conjugate according to claim 15, wherein X₁, X₂, X₃, X₄, and G each represent a hydrogen atom.

17: A method for producing a conjugate in which a desired molecule is bound to an indole structure in an indole-structure-containing molecule, the method comprising cross-linking the indole-structure-containing molecule to the desired molecule through a compound having an N-oxy radical group and a group capable of bonding to the desired molecule.

18: The on method according to claim 17, further comprising allowing the desired molecule to act on the compound, and then allowing the compound, to which the desired molecule is bound, to act on the indole structure in the indole-structure-containing molecule.

19: The method according to claim 17, further comprising allowing the compound to act on the indole structure in the indole-structure-containing molecule, and then allowing the desired molecule to act on the compound bound to the indole-structure-containing molecule.

20: The method according to claim 17, wherein the cross-linking reaction is carried out in an aqueous solvent.
